(19) United States
(12) Patent Application Publication
(10) Pub. No.: US 2023/0313180 A1
(43) Pub. Date: Oct. 5, 2023

(54) MULTIMERIC ANTIBODY PLATFORM AND METHODS OF MAKING AND USING MULTIMERIC ANTIBODY PLATFORM

(71) Applicant: Oregon State University, Corvallis, OR (US)

(72) Inventors: Ryan A. Mehl, Corvallis, OR (US); Elise M. Van Fossen, Corvallis, OR (US); Riley M. Bednar, Corvallis, OR (US)

(73) Assignee: Oregon State University, Corvallis, OR (US)

(21) Appl. No.: 18/061,389

(22) Filed: Dec. 2, 2022

Related U.S. Application Data
(60) Provisional application No. 63/285,315, filed on Dec. 2, 2021.

(51) Int. Cl. C12N 15/11; C12N 9/00; C07K 16/10

(52) U.S. Cl. CPC C12N 15/11; C12N 9/93; C12Y 601/01018; C12Y 601/01014; C07K 16/10; C07K 2317/569; C07K 2317/24

(57) ABSTRACT

Embodiments of the claimed invention describe methods for making a multimeric nanobody assembly. In some embodiments, a method for making a multimeric nanobody assembly comprises providing polynucleic acids that encode for nanobodies, wherein the polynucleic acids each further comprise at least one selector codon at a preselected position, providing a non-peptide linker comprising a strained alkene functional group, and providing a translational system, wherein the polynucleic acids are translated by the translational system to encode the nanobodies, and wherein the tetrazine amino acids react with the strained alkene functional group to produce the multimeric nanobody assembly.

### CROSS-REFERENCE TO RELATED APPLICATION

This application claims the benefit of U.S. Provisional Application No. 63/285,315, filed Dec. 2, 2021, the disclosure of which is incorporated herein by reference in its entirety.

### STATEMENT OF GOVERNMENT LICENSE RIGHTS

This invention was made with Government support under Grant No. R01 GM131168 and awarded by the National Institutes of Health and Grant Nos. MCB1518265 and MCB0448297 awarded by the National Science Foundation. The Government has certain rights in the invention.

### STATEMENT REGARDING SEQUENCE LISTING

The Sequence Listing XML associated with this application is provided in XML format and is hereby incorporated by reference into the specification. The name of the XML file containing the sequence listing is 3014-P23US_Seq_List_20221202.xml. The XML file is 28 KB; was created on Dec. 2, 2022; and is being submitted via Patent Center with the filing of the specification.

### BACKGROUND

Single-domain antibodies, commonly referred to as nanobodies (Nbs), are small (˜15 kDa) protein binders derived from Camelidae antibodies. Nbs are stable, easy to produce, highly modifiable alternatives to conventional antibodies, and are often used as therapeutics, diagnostics, imaging agents and tools for structural biology studies. The generation of multivalent Nb assemblies from Nb monomers has proven powerful for generating Nb-based therapeutics, as functionalization can provide higher avidity than their monomeric counterparts as well as longer lifetimes and the ability to bind multiple, diverse targets. More recently, the multimerization of Nbs targeted against SARS-CoV-2 have shown to enhance their abilities both as binders and therapeutics against viruses. As the demand for Nb multimerization and functionalization increases, so will the need for conjugation strategies that can multimerize and functionalize Nbs without impacting Nb binding to its target.

The binding potential of Nbs stems from three complementarity-determining regions (CDR) supported by conserved framework comprised of a β-sheet-based domain. While all three CDR loops contribute to target epitope binding, the CDR3 loop possesses the highest variability with respect to loop length and position, as it can protrude outward from the N-terminus or fold over onto the side of the domain when participating in target binding. Because of the potential length, location and variability of the CDR loops, care must be taken to functionalize nanobodies in a manner that will not impede their target binding.

This is typically accomplished by genetic fusions, either to another Nb in tandem, or by utilizing dimerization domains. These strategies both restrict the Nb conjugation sites to the N- or C-termini, which, because of their close proximity to CDR regions, can be deleterious for binding interactions and also for tandem arrangements, require linker length and Nb order optimization as one CDR region can be blocked by the genetic fusion of the other nanobody. Additionally, the utilization of the terminal ends for conjugation, even if binding is not impacted, prevents the terminal usage for PEGylation, labeling or drug conjugation.

Site-specific functionalization with non-canonical amino acids (ncAA) that are not limited to protein termini are possible with genetic code expansion (GCE), have been successfully implemented in large molecules intended for therapeutic usage, including antibody conjugation and PEGylation; however, Nb functionalization with GCE as intended for therapeutic utility remains limited. This may be that because the strategies employed with antibody functionalization to generate a substantial amount of chemically pure product (excess of reagents to drive the reactions, long coupling times) are not appropriate for the smaller Nb, as the impact of inactivating key residues through lengthy or off-target labeling reactions for Nbs becomes more consequential and it is more challenging to resolve functional products from unreactive material and side-reactions. As such, successful Nb conjugations intended for therapeutic use require approaches that not only (1) grant freedom of attachment but also (2) utilize high yield efficacious and specific labeling reactions that occur under biologically compatible time frames and conditions.

Recently, the inventors have developed a number of GCE systems for incorporating tetrazine-containing ncAAs that undergo ultrafast reactions (k₂˜8×10⁴ M⁻¹ s⁻¹) with strained trans-cyclooctenes, allowing the achievement of sub-stoichiometric labeling of tetrazine-containing proteins, even within living cells. This reaction speed, along with improved suppression ability offers the flexibility to incorporate tetrazine at even well-conserved sites in a polypeptide that are either not permissive to mutagenesis or amenable to chemical conjugation. The inventors determined that this GCE-enabled bioorthogonal reaction can lead to more complex Nb assemblies with increased functionality by allowing precision conjugations to be performed at previously under-utilized surface amino acid residues.

Here, in addition to a general description of the methods and compositions, specific embodiments are disclosed that improve on the prior art to generate multimeric Nb assemblies that, for example, can function as anti-SARS-CoV-2 therapeutic agents that target the receptor binding domain (RBD) of the SARS-CoV-2 spike protein, including specifically targeting the trimeric organization of the spike protein and targeting independent RBD epitopes so as to prevent viral escape. By site-specifically encoding a reactive tetrazine ncAA at multiple positions on existing anti-RBD Nbs, it has been demonstrated that the speed and robustness of the GCE-encoded tetrazine coupling reaction removes the concern that conjugation conditions will compromise protein function, resulting in the generation of anti-SARS-CoV-2 homodimeric Nbs with improved binding as well as the generation of Nb trimers and heterodimers with structure-determined conjugation points with enhanced neutralization ability.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In accordance with the foregoing, in one aspect of the invention, the disclosure provides method of making a multimeric nanobody assembly, the method comprising: providing at least two polynucleotides encoding at least two nanobodies, wherein the polynucleotides each further comprise at least one selector codon at a preselected position; providing a non-peptide linker comprising a strained alkene functional group; and providing a translational system, wherein the at least two polynucleotides are translated by the translational system to produce the at least two nanobodies comprising a tetrazine amino acid, and wherein the translational system further includes: at least one tetrazine amino acid; an orthogonal tRNA that decodes the selector codon; an orthogonal aminoacyl-tRNA synthetase that charges the orthogonal tRNA with the tetrazine amino acid; and wherein the orthogonal tRNA inserts the tetrazine amino acid into a polypeptide in response to the selector codon, wherein the polypeptide comprises the nanobody comprising the incorporated tetrazine amino acid; and wherein the at least two tetrazine amino acids react with the non-peptide linker terminated by the strained alkene functional group to produce the multimeric nanobody assembly.

In accordance with the foregoing, in another aspect of the invention, the disclosure provides a multimeric nanobody assembly, the nanobody assembly comprising: a plurality of nanobodies specific for a target ligand, wherein the nanobodies are encoded with one or more tetrazine amino acid; and a non-peptide linker comprising a strained alkene functional group associated with a tetrazine amino acid; wherein the multimeric nanobody assembly is functionalized by the covalent association of the tetrazine amino acid and the non-peptide linker such that each nanobody of the multimeric nanobody assembly retains its binding specificity to its target ligand.

In accordance with the foregoing, in another aspect of the invention, the disclosure provides a of inhibiting virus infectivity, the method comprising administering a therapeutically effective quantity of a pharmaceutical composition to a subject in need thereof, wherein the pharmaceutical composition comprises an active ingredient that inhibits virus infectivity and a pharmaceutically acceptable excipient, wherein the active ingredient is the multimeric nanobody assembly described above.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A through 1D. Structural features of Nbs and various assembly strategies. FIG. 1A. Ribbon diagram of Nb1 (VHH72, PDB_ID 6WAQ) as a representative Nb, highlighting the three CDRs, the N-terminus, the C-terminus and framework sites Gln13 (Q13) and Gly42 (G42). Shown to the right are front, back and top views of thirty (30) overlaid Nb structures from the PDB. FIG. 1B. Three common Nb dimerization modes are shown using a generic cartoon Nb domain: (i) “tail to head” genetic conjugation; (ii) dimerization domain-mediated and (iii) sortase A-mediated. FIG. 1C. Ribbon diagrams showing distinct SARS-CoV-2 RBD epitopes recognized by Nb1 (PDB_ID 6ZH9) and Nb2 (PDB_ID 6WAQ). ACE2 is also shown as it binds to the SARS-CoV-2 RBD (PDB_ID 6MOJ). A dashed line connects the Nb1 N-terminus and Nb2 residue 42 positions that will be used to link the Nbs. FIG. 1D. Nb and PEG-linker components used in this study, with various positions of Tet3.0 incorporation for Nb1 and Nb2 indicated by stars, and sTCO-functionalized groups on the PEG linkers indicated by circled stars. Images and overlays generated by Chimera.

FIGS. 2A through 2D. Expression of tet-encoded Nbs and validation of tet-reactivity. FIG. 2A. Constructs used for Nb1 and Nb2 expression are shown in the boxes and the mature protein products derived from each are on the right after the arrows. Indicated are the sites of amber codons for each construct (stripes) and the positions of Tet3.0 in the mature proteins (stars). FIG. 2B. 15 SDS-PAGE gel of molecular weight standards and purified Nb monomers (migrating at ˜15 kDa) with labels above each lane. FIG. 2C. ESI MS analyses of Nb1 monomer and Nb1_{N}. Observed (arrows) and expected masses are listed and the change in side-chain at the N-terminal position (Gln vs. Tet3.0) is shown with the expected mass shift. FIG. 2D. SDS-PAGE gel showing molecular weight standards and the mobility of each Tet3.0-containing Nb in the absence (“−”) and presence (“+”) of sTCO- with PEG₅ₖ as noted above each lane.

FIGS. 3A through 3D. Evaluation of the effect of point of conjugation on SARS-COV RBD interaction by Nb1 dimers. FIG. 3A. Homo-dimeric conjugations of Nb1 generated in this study, with points of conjugation indicated by stars. FIG. 3B. 4-20% gradient SDS-PAGE gel of purified Nb homodimers as labeled above each lane. FIG. 3C. Sensograms for BLI analysis of conjugated Nbs with immobilized SARS-CoV-2 RBD. Concentrations on conjugated Nbs ranged from 2 nM-1500 nM for each assay and global fits were obtained by fitting the sensograms to a 1:2 binding curve (light gray line). Only fits with an R2 value greater than 0.98 were considered acceptable. FIG. 3D. K_{D} values plotted for all conjugated Nbs, the Nb corresponding to the K_{D} is indicated under the plot.

FIGS. 4A through 4F. Generation and evaluation of GCE-conjugated Nb multimers. FIG. 4A. Multimeric conjugations of Nbs generated in this study, with points of conjugation indicated by stars. FIG. 4B. 4-20% gradient SDS-PAGE gel of purified Nb multimers as labeled above each lane. “**” indicates the expected migration of the heterodimer at ˜30 kDa and “****” indicates expected migration of the trimer at ˜60 kDa. Due to the size and topography of the Tri-armed, 15 kDa PEG linker, the product resolves as a broad band. FIG. 4C. Top: Mass spectrometry results for (Nb1_{N})₃ trimer. The presence of PEG-separated (+88 Da) peaks at an average mass of ˜58 kDa agrees with the expected mass of a triply-conjugated Nb 15 kDa PEG multimer (58715.5 Da). FIG. 4D. Sensograms for BLI analysis of conjugated Nbs with immobilized SARS-CoV-2 RBD. Concentrations on conjugated Nbs ranged from 2 nM-1500 nM for each assay and global fits were obtained by fitting the sensograms to a 1:1 binding curve (light gray line). Only fits with an R2 value greater than 0.98 were considered acceptable. FIG. 4E. K_{D} values plotted for all conjugated Nbs, the Nb corresponding to the K_{D} is indicated under the plot. FIG. 4F. Percent infectivity of a SARS-CoV-2 pseudovirus for three Nb conjugates, plotted on a Log/Log plot. Percent infectivity was determined by calculating the percent decrease in luciferase signal from 293T-hACE2 cells infected with Nb-treated SARS-CoV-2 pseudovirus (EXPT column, filled in shapes) compared to buffer-treated SARS-CoV-2 pseudovirus (triangle). The percent infectivity of the Nb conjugate set incubated with VSG pseudovirus without spike protein (CTRL) is indicated by muted lines and markers. Data points of the EXPT values that represent a statistically significant differences (p<0.5) from the CTRL values are indicated with asterisks (ANOVA: single factor (p −0.020, p −0.006).

FIGS. 5A and 5B. NMR analysis of tetrazines. FIG. 5A. ¹H NMR (400 MHz, CD3OD). FIG. 5B. ¹³C NMR (175 MHz, CD3OD).

FIGS. 6A and 6B. NMR analysis for sTCO. FIG. 6A. ¹H NMR (700 MHz, CD3OD). FIG. 6B. ¹H NMR (400 MHz, CDCl₃) of activated ester.

FIG. 7. NMR analysis of PEG550 linked di-sTCO ¹H NMR (400 MHz, CD3OD) for sTCO-PEG12.

FIG. 8. NMR analysis of 3-Arm-PEG15k linked tri-sTCO ¹H NMR (400 MHz, CDCl3) for sTCO-PEG15 kDa.

FIGS. 9A through 9C. Electron Capture Dissociation in tandem with mass spectrometry. FIG. 9A. Sequence map depicting the ion fragments detected from Nb1N. The site of tetrazine incorporation was identified in position 6 by the detection of a 113.04 Da increase in mass compared to the wild type. The outer circles indicate c and z ions, and the inner circles indicate b and y ions. The top-down analysis yielded an overall sequence coverage of 56% for the tetrazine-containing nanobody. FIGS. 9B and 9C. Representative mass spectra of the tetrazine containing nanobody. The upper panel shows the numerous fragments matched in the ECD spectrum while the lower panel displays a zoomed-in portion that shows the matching of individual fragment ions. (SGTGS) SEQ ID NO: 26; (VQLQESGGGLVQAGGSLRLSCAASGRTFSEYAMGWFRQAPGKEREFVATISWS GGSTYYTDSVKGRFTISRDNAKNTVYLQMNSLKPDDTAVYYCAAAGLGTVVSE WDYDYYLDYWGQGTQVTVSS) SEQ ID NO: 27.

FIGS. 10A through 10E. Generation and purification of homodimers. FIG. 10A. Ratios of Tet3.0-Nb and di-sTCO-PEG12 tested are listed at the top of the gel. “*” indicates unreacted Tet3.0-Nb (˜15 kDa) and “**” indicates homodimers (˜30 kDa). FIGS. 10B through 1E. Reaction products (“input”) were resolved by size exclusion chromatography (SEC). “*” indicates unreacted Tet3.0-Nb (˜15 kDa) and “**” indicates the homodimer products (˜30 kDa). Fractions are indicated at the top of the gel and the collected fractions are indicated with a black box.

FIGS. 11A through 11C. Generation and purification of heterodimer. FIG. 11A. Potential products formed after incubation of di-sTCO-PEG12 with Tet3.0-Nb1 and Tet3.0-Nb2-TEV-GFP-His. FIG. 11B. SEC analysis of heterodimeric conjugation reaction. Heterodimeric products (shaded area), as determined by 2=495 nm to detect GFP chromophore (bottom trace) and hydrodynamic radius (top trace) were analyzed with SDS-PAGE. FIG. 11C. SDS-PAGE analysis of collected fractions. Starting materials and reaction mixtures are indicated by “−” and “+” di-sTCO PEG12 respectively. Products are indicated with boxes (3rd column) and starting material are indicated with boxes (2nd column). Fractions that were combined for further analysis are indicated with a large rectangle.

FIGS. 12A through 12D. Generation and purification of trimer. FIG. 12A. Determining the correct ratio of Tri-sTCO PEG and Nb1N. The formation of trimer is determined by the disappearance of starting material (single asterisk) and the appearance of higher MW products (triple asterisks). FIG. 12B. SEC analysis of trimeric conjugation reaction. Trimeric products (indicated with first bracket) were resolved from unreacted materials (second bracket) and were analyzed with SDS-PAGE. FIG. 12C. SDS-PAGE analysis of collected fractions. Starting materials and reaction mixtures are indicated by “−” and “+” di-sTCO PEG12 respectively. Fractions that were combined for further analysis are indicated with a black box. FIG. 12D. Mass spectrometry results for (Nb1N)3 trimer. The presence of PEG-separated (+88 Da) peaks at an average mass of 58 kDa agrees with the expected mass of a triply conjugated Nb 15 kDa PEG multimer (58715.5 Da).

FIGS. 13A through 13C. Pseudovirus neutralization assay. Raw luminescence counts for 293T-hACE2cells, infected with nanobody conjugate-treated, SARS-COV2 spike coated pseudovirus (FIG. 13A), VSG pseudovirus (FIG. 13B) and a bald lentiviral pseudovirus which was produced without envelope glycoproteins such as VSV-G or SARS-CoV-2 spike (FIG. 13C). Assays were performed in triplicate.

### DETAILED DESCRIPTION

Assembling nanobodies (Nb) into polyvalent multimers is a powerful strategy for improving the effectiveness of Nb-based therapeutics and biotechnological tools. However, generally effective approaches to Nb assembly are currently restricted to the N- or C-termini, greatly limiting the diversity of Nb multimers that can be produced. This disclosure describes that reactive tetrazine groups—site-specifically inserted by genetic code expansion (GCE) at Nb surface sites—are compatible with Nb folding and function. Using two anti-SARS-CoV-2 Nbs with therapeutic potential, suitable linkers are used to create Nb homo- and heterodimers with improved properties compared with conventionally linked Nb homodimers. Also disclosed are embodiments describing how an even higher affinity homotrimer can be created that shows enhanced viral neutralization. Thus, this tetrazine-based approach is a generally applicable strategy that greatly increases the diversity of accessible Nb-assemblies and adds assembly geometry as something that can be easily varied to optimize the properties of Nb-assemblies.

Disclosed herein are embodiments of a product and a method for assembling nanobodies (Nb) into polyvalent multimers to improve the effectiveness of Nb-based therapeutics and biotechnological tools.

In one aspect, the disclosure provides a method of making a multimeric nanobody assembly. In some embodiments, the method can comprise providing at least two polynucleotides encoding at least two nanobodies, wherein the polynucleotides each further comprise at least one selector codon at a preselected position; providing a non-peptide linker comprising a strained alkene functional group; and providing a translational system, wherein the polynucleotides are translated by the translational system to produce the nanobodies comprising a tetrazine amino acid.

A “multimeric nanobody assembly” refers to a nanobody construct comprising a plurality of nanobodies. In some embodiments, the multimeric nanobody assembly comprises a dimer, a trimer, a tetramer, a pentamer, a hexamer, a heptamer, an octamer, a nonamer, or a decamer. In other embodiments, the multimer nanobody assembly can be a homomultimer comprising a plurality of the same nanobodies. In other embodiments, the multimer nanobody assembly can be a heteromultimer comprising a plurality of different nanobodies. For example, the heteromultimer can be a heterotrimer, which can include but is not limited to three different nanobodies. In other embodiments, for example, the heterotrimer can comprise two of the same nanobodies and one different nanobody.

In some embodiments, a nanobody comprises a variable region of a heavy chain of an antibody and constructed as a single domain antibody (VHH) consisting of only one heavy chain variable region. It is the smallest antigen-binding fragment with complete function.

As used herein, the terms “single domain antibody (VHH)” and “nanobody or nanobodies” have the same meaning referring to a variable region of a heavy chain of an antibody construct and a single domain antibody (VHH) consisting of only one heavy chain variable region. It is the smallest antigen-binding fragment with complete function. Generally, the antibodies with a natural deficiency of the light chain and the heavy chain constant region 1 (CH1) are first obtained, the variable regions of the heavy chain of the antibody are therefore cloned to construct a single domain antibody (VHH) consisting of only one heavy chain variable region.

As used herein, the term “variable” refers that certain portions of the variable region in the nanobodies vary in sequences, which forms the binding and specificity of various specific antibodies to their particular antigen. However, variability is not uniformly distributed throughout the nanobody variable region. It is concentrated in three segments called complementarity-determining regions (CDRs) or hypervariable regions in the variable regions of the light and heavy chain. The more conserved part of the variable region is called the framework region (FR). The variable regions of the natural heavy and light chains each contain four FR regions, which are substantially in a β-folded configuration, joined by three CDRs which form a linking loop, and in some cases can form a partially β-folded structure. The CDRs in each chain are closely adjacent to the others by the FR regions and form an antigen-binding site of the nanobody with the CDRs of the other chain (see e.g., Kabat et al., NIH Publ. No. 91-3242, Volume I, pages 647-669. (1991)). The constant regions are not directly involved in the binding of the nanobody to the antigen, but they exhibit different effects or functions, for example, involve in antibody-dependent cytotoxicity of the antibodies. As used herein, the term “heavy chain variable region” and “V_{H}” can be used interchangeably. As used herein, the terms “variable region” and “complementary determining region (CDR)” can be used interchangeably. In other embodiments, the heavy chain variable region of said nanobody comprises 3 complementary determining regions: CDR1, CDR2, and CDR3.

The term “polynucleic acid” generally refers to any polyribonucleotide or polydeoxyribonucleotide, which may be unmodified RNA or DNA or modified RNA or DNA. Thus, for instance, polynucleic acid as used herein refers to, among others, single and double stranded DNA, DNA that is a mixture of single and double stranded regions, single and double stranded RNA, and RNA that is mixture of single and double stranded regions, hybrid molecules comprising DNA and RNA that may be single stranded or, more typically, double stranded or a mixture of single and double stranded regions. In addition, polynucleic acid as used herein refers to triple stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple helical region often is an oligonucleotide.

In some embodiments, the polynucleotides are heterologous polynucleotides. In other embodiments, the polynucleotides are homologous polynucleotides. In still other embodiments, the polynucleotides are a combination of heterologous and homologous polynucleotides. In some embodiments, the method comprises at least two polynucleotides, at least three polynucleotides, at least four polynucleotides, or 5 or more polynucleotides.

As used herein, “single domain antibody (VHH)” and “nanobody or nanobodies” specifically recognize and bind to the receptor binding domain of the SARS-CoV-2 spike protein. In one embodiment, the nanobody is VHH72, which is referred to as Nb1. In another embodiment, the nanobody is H11-H4, which is referred to as Nb2.

In some embodiments, the translational system further includes at least one tetrazine amino acid; an orthogonal tRNA that decodes the selector codon; an orthogonal aminoacyl-tRNA synthetase that charges the orthogonal tRNA with the tetrazine amino acid; and wherein the orthogonal tRNA inserts the tetrazine amino acid into a polypeptide in response to the selector codon, wherein the polypeptide comprises the nanobody comprising the incorporated tetrazine amino acid; and wherein the at least two tetrazine amino acids react with the non-peptide linker terminated by the strained alkene functional group to produce the multimeric nanobody assembly.

The translation system uses the tRNA/aaRS pair to incorporate a tetrazine amino acid into a growing polypeptide chain, where the nucleic acid comprises a selector codon that is recognized by tRNA. An anticodon loop of the tRNA (CUA) can recognize the selector codon on mRNA and incorporate its tetrazine amino acid at the corresponding site in the growing polypeptide.

The term “translation system” refers to the components that incorporate an amino acid into a growing polypeptide chain (protein). Components of a translation system can include, e.g., ribosomes, tRNAs, synthetases, mRNA, and the like. Typical translation systems include cells, such as bacterial cells (e.g., Escherichia coli), archeaebacterial cells, eukaryotic cells (e.g., yeast cells, mammalian cells, plant cells, insect cells), or the like. Alternatively, the translation system comprises an in vitro translation system, e.g., a translation extract including a cellular extract. The O-tRNA or the O-RSs of the invention can be added to or be part of an in vitro or in vivo translation system, e.g., in a non-eukaryotic cell, e.g., a bacterium (such as E. coli), or in a eukaryotic cell, e.g., a yeast cell, a mammalian cell, a plant cell, an algae cell, a fungus cell, an insect cell, or the like. The translation system can also be a cell-free system, e.g., any of a variety of commercially available in vitro transcription/translation systems in combination with an O-tRNA/O-RS pair and a tetrazine amino acid as described herein.

The translation system can optionally include multiple O-tRNA/O-RS pairs, which allow incorporation of more than one unnatural amino acid, e.g., a tetrazine amino acid at more than one position. For example, the cell can further include an additional different O-tRNA/O-RS pair and a second tetrazine amino acid, where this additional O-tRNA recognizes a second selector codon and this additional O-RS preferentially aminoacylates the O-tRNA with the second tetrazine amino acid. For example, a cell that includes an O-tRNA/O-RS pair (where the O-tRNA recognizes, e.g., an amber selector codon) can further comprise a second orthogonal pair, where the second O-tRNA recognizes a different selector codon at a different position (e.g., an opal codon, four-base codon, or the like). Desirably, the different orthogonal pairs are derived from different sources, which can facilitate recognition of different selector codons.

In general, when an orthogonal pair recognizes a selector codon and loads an amino acid in response to the selector codon, the orthogonal pair is said to “suppress” the selector codon. That is, a selector codon that is not recognized by the translation system's (e.g., cell's) endogenous machinery is not ordinarily translated, which results in blocking production of a polypeptide that would otherwise be translated from the nucleic acid. An O-tRNA of the invention recognizes a selector codon and includes at least about, e.g., a 45%, a 50%, a 60%, a 75%, an 80%, or a 90% or more suppression efficiency in the presence of a complementary synthetase in response to a selector codon as compared to an O-tRNA comprising or encoded by a polynucleotide sequence as set forth herein. The translation system (e.g., a cell) uses the O-tRNA/O-RS pair to incorporate the unnatural amino acid into a growing polypeptide chain, e.g., via a nucleic acid that comprises a polynucleotide that encodes a polypeptide of interest, where the polynucleotide comprises a selector codon that is recognized by the O-tRNA. An anticodon loop of the O-tRNA may recognize the selector codon on an mRNA and incorporate its tetrazine amino acid at the corresponding site in the polypeptide.

Selector codons of the invention expand the genetic codon framework of the protein biosynthetic machinery. For example, a selector codon includes, e.g., a unique three base codon, a nonsense codon, such as a stop codon, e.g., an amber codon (UAG), or an opal codon (UGA), an unnatural codon, at least a four-base codon (e.g., AGGA), a rare codon, or the like. A number of selector codons can be introduced into a desired gene, e.g., one or more, two or more, more than three, and the like. By using different selector codons, multiple orthogonal tRNA/synthetase pairs can be used that allow the simultaneous site-specific incorporation of multiple different unnatural amino acids, using these different selector codons. Similarly, more than one copy of a given selector codon can by introduced into a desired gene to allow the site-specific incorporation of a given unnatural amino acid at multiple sites (e.g., two or more, three or more, etc.). For example, a stop codon can be used as a selector codon for the incorporation of a tetrazine amino acid, in which case an O-tRNA can be produced that recognizes a stop selector codon and is aminoacylated by an O-RS with a tetrazine amino acid.

Conventional site-directed mutagenesis can be used to introduce the selector codon at the site of interest in a target polynucleotide encoding a polypeptide of interest. When the O-RS, O-tRNA and the nucleic acid that encodes a polypeptide of interest are combined, e.g., in vivo, the tetrazine amino acid is incorporated in response to the selector codon to give a polypeptide containing the tetrazine amino acid at the specified position.

The incorporation of tetrazine amino acids in vivo can be done without significant perturbation of the host cell. For example, in non-eukaryotic cells, such as Escherichia coli, because the suppression efficiency of a stop selector codon, e.g., the UAG codon, depends upon the competition between the O-tRNA, e.g., the amber suppressor tRNA, and release factor 1 (RF1), which binds to the UAG codon and initiates release of the growing peptide from the ribosome, the suppression efficiency can be modulated by, e.g., either increasing the expression level of O-tRNA, e.g., the suppressor tRNA, or using an RF1 deficient strain. In eukaryotic cells, because the suppression efficiency for a UAG codon depends upon the competition between the O-tRNA, e.g., the amber suppressor tRNA, and a eukaryotic release factor (e.g., eRF), which binds to a stop codon and initiates release of the growing peptide from the ribosome, the suppression efficiency can be modulated by, e.g., increasing the expression level of O-tRNA, e.g., the suppressor tRNA. In addition, additional compounds can also be present that modulate release factor action, e.g., reducing agents such as dithiothreitol (DTT).

The translational components of the invention can be derived from non-eukaryotic organisms. For example, the orthogonal O-tRNA and O-RSs can be derived from a non-eukaryotic organism (or a combination of organisms), e.g., an archaebacterium, a Eubacterium, or the like. In one embodiment, eukaryotic sources, e.g., plants, algae, protists, fungi, yeasts, animals (e.g., mammals, insects, arthropods, etc.), or the like, can also be used as sources of O-tRNAs and O-RSs. The individual components of an O-tRNA/O-RS pair can be derived from the same organism or different organisms. In one embodiment, the O-tRNA/O-RS pair is from the same organism. Alternatively, the O-tRNA and the O-RS of the O-tRNA/O-RS pair are from different organisms.

Host cells are genetically engineered (e.g., transformed, transduced or transfected) with the polynucleotides of the invention or constructs that include a polynucleotide of the invention, e.g., a vector of the invention, which can be, for example, a cloning vector or an expression vector. For example, the coding regions for the orthogonal tRNA, the orthogonal tRNA synthetase and the protein to be derivatized are operably linked to gene expression control elements that are functional in the desired host cell.

Typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both (e.g., shuttle vectors) and selection markers for both prokaryotic and eukaryotic systems. Vectors are suitable for replication or integration in prokaryotes, eukaryotes, or preferably both. See, Giliman and Smith, Gene 8:81 (1979); Roberts, et al., Nature 328:731 (1987); Schneider, et al., Protein Expr. Purif. 6435:10 (1995). The vector can be, for example, in the form of a plasmid, a bacterium, a virus, a naked polynucleotide, or a conjugated polynucleotide. The vectors are introduced into cells or microorganisms by standard methods including electroporation infection by viral vectors, high velocity ballistic penetration by small particles with the nucleic acid either within the matrix of small beads or particles or on the surface. See, e.g., From, et al., Proc. Natl. Acad. Sci. U.S.A. 82:5824 (1985); Klein, et al., Nature 327:70-73 (1987).

The engineered host cells can be cultured in conventional nutrient media modified as appropriate for such activities as, for example, screening steps, activating promoters or selecting transformants These cells can optionally be cultured into transgenic organisms.

As used herein, “selector codon” refers to a codon recognized by the orthogonal tRNA in the translation process and not typically recognized by an endogenous tRNA. The O-tRNA anticodon loop recognizes the selector codon on the mRNA and incorporates its amino acid, e.g., a tetrazine amino acid, at this site in the polypeptide. Selector codons can include, e.g., nonsense codons, such as stop codons (e.g., amber, ochre, and opal codons), four or more base codons, rare codons, codons derived from natural or unnatural base pairs, or the like. Selector codons can also comprise extended codons, e.g., four or more base codons, such as four, five, six, or more base codons. Examples of four base codons include, e.g., AGGA, CUAG, UAGA, CCCU, and the like. Examples of five base codons include, e.g., AGGAC, CCCCU, CCCUC, CUAGA, CUACU, UAGGC, and the like.

The term “orthogonal” as used herein refers to a molecule, e.g., an orthogonal tRNA (O-tRNA) or an orthogonal aminoacyl-tRNA synthetase (O-RS) that functions with endogenous components of a cell or other translation system with reduced efficiency as compared to a corresponding molecule that is endogenous to the cell or translation system or that fails to function when paired with endogenous components of the cell or translation system. In the context of tRNAs and aminoacyl-tRNA synthetases, orthogonal refers to an inability or reduced efficiency (e.g., less than 20% efficiency, less than 10% efficiency, less than 5% efficiency, or less than 1% efficiency) of an orthogonal tRNA to function with an endogenous tRNA synthetase compared to the ability of an appropriate (e.g., homologous or analogous) endogenous tRNA to function when paired with the endogenous complementary tRNA synthetase; or of an orthogonal aminoacyl-tRNA synthetase to function with an endogenous tRNA as compared to the ability of an appropriate endogenous tRNA synthetase to function when paired with the endogenous complementary tRNA.

An “orthogonal” molecule lacks a functionally normal, naturally occurring endogenous complementary molecule in the cell or translation system. For example, an orthogonal tRNA in a cell is aminoacylated by any endogenous RS of the cell with reduced or even undetectable efficiency, when compared to aminoacylation of an endogenous tRNA by the endogenous RS. In another example, an orthogonal RS aminoacylates any endogenous tRNA in a cell of interest with reduced or even undetectable efficiency, as compared to aminoacylation of the endogenous tRNA by a complementary endogenous RS. A second orthogonal molecule can be introduced into the cell that functions when paired with the first orthogonal molecule. For example, an orthogonal tRNA/RS pair includes introduced complementary components that function together in the cell with an efficiency (e.g., 45% efficiency, 50% efficiency, 60% efficiency, 70% efficiency, 75% efficiency, 80% efficiency, 90% efficiency, 95% efficiency, or 99% or more efficiency) as compared to that of a control, e.g., a corresponding (e.g., analogous) tRNA/RS endogenous pair, or an active orthogonal pair.

An “orthogonal tRNA” (O-tRNA) as used herein is a tRNA that is orthogonal to a translation system of interest. The O-tRNA can exist charged with an amino acid or in an uncharged state. It will be appreciated that an O-tRNA of the invention is advantageously used to insert essentially any amino acid, whether natural or unnatural, into a growing polypeptide, during translation, in response to a selector codon. An orthogonal tRNA of the invention desirably mediates incorporation of a tetrazine amino acid into a protein that is encoded by a polynucleotide that comprises a selector codon that is recognized by the O-tRNA.

As used herein, “orthogonal aminoacyl-tRNA synthetase” (O-RS) is an enzyme that preferentially aminoacylates an O-tRNA with an amino acid in a translation system of interest.

As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term “encode” describes the process of semi-conservative DNA replication, where one strand of a double-stranded DNA molecule is used as a template to encode a newly synthesized complementary sister strand by a DNA-dependent DNA polymerase. In another aspect, the term “encode” refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. When used to describe the process of translation, the term “encode” also extends to the triplet codon that encodes an amino acid. In some aspects an RNA molecule can encode a DNA molecule, e.g., by the process of reverse transcription incorporating an RNA-dependent DNA polymerase. In another aspect, a DNA molecule can encode a polypeptide, where it is understood that “encode” as used in that case incorporates both the processes of transcription and translation.

As used herein, a non-peptide linker is used to connect multiple nanobodies through a reactive tetrazine group site-specifically inserted by genetic code expansion on each nanobody enabling assembly of the nanobody platform. In some embodiments, the non-peptide linker comprises a 1,4-dihydropyridazine linkage or a dihydropyridazine linkage. In other embodiments, the strained alkene functional group is a trans-cycloctene (TCO) functional group, or a derivative thereof. In still other embodiments, the TCO or derivative thereof, comprises a 2-headed sTCO polyethylene glycol₁₂ (PEG)₁₂ linker or a 3-armed Tri-sTCO (PEG)_{3×5k} linker.

### TCO-PEG Linkers

In some embodiments, TCO PEG linkers (reagents) are a class of PEG linkers with PEG attached to a TCO group (trans-cycloctene), TCO reagent is highly reactive with tetrazine in an inverse electron demand Diels Alder (IEDDA) reaction followed by a retro-Diels Alder reaction to eliminate nitrogen gas. This is a fast reaction with a second order rate constant of 2000 M⁻¹ s⁻¹ (in 9:1 methanol/water) allowing modification of biomolecules at very low concentrations. Reactions have also been performed using TCO or norbornenes as dienophiles at second order rates on the order of 1 M⁻¹ s⁻¹ in aqueous media. This reaction has been applied in labeling live cells, molecular imaging and other bioconjugation applications.

In some embodiments, the method comprises introducing at least one tetrazine amino acid at a specific site in a nanobody using the method described above to create a tetrazine modified nanobody. One of ordinary skill in the art would appreciate that tetrazine amino acids in proteins reach reaction rates of 8×10⁴ M⁻¹ s⁻¹ with sTCO reagents. Additionally, one of ordinary skill in the art would appreciate that using the method described above, and further described in the Example Section, reactive tetrazine groups can be site-specifically inserted at any desired nanobody surface site. As such, embodiments of the claimed method allow for generating rapid nanobody assemblies with fully flexible topology.

In some embodiments, the multimeric nanobody assembly is a homomultimer. In some embodiments, the multimeric nanobody assembly is a homodimer, wherein each monomer expresses at least one tetrazine amino acid at any position along the polypeptide sequence. In other embodiments, the multimeric nanobody assembly is a homotrimer, wherein each monomer expresses at least one tetrazine amino acid at any position along the polypeptide sequence.

In still other embodiments, the multimeric nanobody assembly is a heteromultimer. In some embodiments, the multimeric nanobody assembly is a heterodimer, wherein each monomer expresses at least one tetrazine amino acid at any position along the polypeptide sequence. In still other embodiments, the multimeric nanobody assembly is a heterotrimer, wherein each monomer expresses at least one tetrazine amino acid at any position along the polypeptide sequence.

In still other embodiments, the multimeric nanobody assembly is a homomultimer comprising at least two polynucleic acids each encoding nanobody VHH72 (Nb1). In still other embodiments, the Nb1 comprises the selector codon designed for incorporating the tetrazine amino acid at a position comprising glutamine 1 (Q1), glutamine 13 (Q13), glycine 42 (G42), or serine 129 (S129). In still other embodiments, the first Nb1 polynucleic acid comprises the selector codon designed for incorporating the tetrazine amino acid at position glutamine 13 (Q13) and the second Nb1 polynucleic acid comprises the selector codon designed for incorporating the tetrazine amino acid at position glycine 42 (G42), wherein Nb1 polypeptide 1 (Nb1)₁ and Nb1 polypeptide 2 (Nb1)₂ assemble as a homodimer in the presence of the strained alkene functional group.

In still other embodiments, the multimeric nanobody assembly is a heteromultimer comprising a polynucleic acid encoding nanobody VHH72 (Nb1) and a polynucleic acid encoding nanobody H11-H4 (Nb2). In some embodiments, the Nb1 comprises the selector codon designed for incorporating the tetrazine amino acid at a position comprising glutamine 1 (Q1), glutamine 13 (Q13), glycine 42 (G42), or serine 129 (S129) and the Nb2 comprises the selector codon designed for incorporating the tetrazine amino acid at a position comprising glycine 42 (G42). In some embodiments, the Nb1 polynucleic acid comprises a selector codon designed for incorporating the tetrazine amino acid at position glutamine 1 (Q1), and the Nb2 polynucleic acid comprises a selector codon designed for incorporating the tetrazine amino acid at position glycine 42 (G42), wherein Nb1 polypeptide and Nb2 polypeptide assemble as a heterodimer in the presence of the strained alkene functional group.

In another aspect, the disclosure provides a multimeric nanobody assembly. In some embodiments, the nanobody assembly can comprise a plurality of nanobodies specific for a target ligand and a non-peptide linker that can comprise a strained alkene functional group, wherein the nanobodies can functionalize with the non-peptide linker such that each nanobody retains its binding specificity to its target ligand.

In another aspect, the disclosure provides a method for inhibiting virus infectivity, the method can comprise administering a therapeutically effective quantity of a pharmaceutical composition to a subject in need thereof, wherein the pharmaceutical composition can comprise an active ingredient that inhibits virus infectivity and a pharmaceutically acceptable excipient, wherein the active ingredient can comprise the multimeric nanobody assembly.

As used herein, the phrase “inhibiting virus infectivity” refers to blocking viral entry into the cell though binding of the virus and preventing its entry into the host cell, thereby reducing viral replication within the host cell as compared to a host cell that was not administered the multimeric nanobody assembly.

In some embodiments, the nanobody assembly used as the active ingredient in the pharmaceutical composition can comprise a plurality of nanobodies specific for a target ligand and a non-peptide linker that can comprise a strained alkene functional group, wherein the nanobodies can functionalize with the non-peptide linker such that each nanobody retains its binding specificity to its target ligand.

In still other embodiments, the pharmaceutical composition can be formulated to be administered using a nebulizer. In some embodiments, the pharmaceutical composition can be formulated to be administered using a nasal spray. In some embodiments, the virus can be a coronavirus. In some embodiments, the coronavirus can be a SARS-CoV-2 coronavirus.

In some embodiments, methods are disclosed to administer a therapeutically effective quantity of a pharmaceutical composition to a subject, wherein the active ingredient of the pharmaceutical composition is a multimeric nanobody assembly. In some embodiments, the multimeric nanobody assembly can be formulated in non-toxic, inert, and pharmaceutically acceptable aqueous carrier media wherein the pH is generally about 5 to 8, preferably about 6 to 8, although the pH can be varied with the nature of the formulation material and the condition to be treated. The formulated pharmaceutical compositions can be administered by conventional routes including, a nebulizer or a nasal spray. In some embodiments, the pharmaceutical composition can be formulated as a powdered formulation and delivered by a nebulizer. One of ordinary skill in the art would formulate the pharmaceutical composition according to methods well known in the art. In some embodiments, the pharmaceutical composition can be formulated in a liquid form and administered as a nasal spray. One of ordinary skill in the art would formulate the pharmaceutical composition according to methods well known in the art.

### Additional Definitions

Unless specifically defined herein, all terms used herein have the same meaning as they would to one skilled in the art of the present invention. Practitioners are particularly directed to Sambrook J., et al., (eds.), Molecular Cloning: A Laboratory Manual, 3rd ed., Cold Spring Harbor Press, Plainsview, New York (2001); Ausubel, F. M., et al., (eds.), Current Protocols in Molecular Biology, John Wiley & Sons, New York (2010); Ran, F. A., et al., Genome engineering using the CRISPR-Cas9 system, Nature Protocols, 8:2281-2308 (2013), and Jiang, F. and Doudna, J. A., CRISPR-Cas9 Structures and Mechanisms, Annual Review of Biophysics, 46:505-529 (2017) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

As used herein, the term “nucleic acid” refers to a polymer of nucleotide monomer units or “residues”. The nucleotide monomer subunits, or residues, of the nucleic acids each contain a nitrogenous base (i.e., nucleobase) a five-carbon sugar, and a phosphate group. The identity of each residue is typically indicated herein with reference to the identity of the nucleobase (or nitrogenous base) structure of each residue. Canonical nucleobases include adenine (A), guanine (G), thymine (T), uracil (U) (in RNA instead of thymine (T) residues) and cytosine (C). However, the nucleic acids of the present disclosure can include any modified nucleobase, nucleobase analogs, and/or non-canonical nucleobase, as are well-known in the art. Modifications to the nucleic acid monomers, or residues, encompass any chemical change in the structure of the nucleic acid monomer, or residue, that results in a noncanonical subunit structure. Such chemical changes can result from, for example, epigenetic modifications (such as to genomic DNA or RNA), or damage resulting from radiation, chemical, or other means. Illustrative and nonlimiting examples of noncanonical subunits, which can result from a modification, include uracil (for DNA), 5-methylcytosine, 5-hydroxymethylcytosine, 5-formethylcytosine, 5-carboxycytosine b-glucosyl-5-hydroxy-methylcytosine, 8-oxoguanine, 2-amino-adenosine, 2-amino-deoxyadenosine, 2-thiothymidine, pyrrolo-pyrimidine, 2-thiocytidine, or an abasic lesion. An abasic lesion is a location along the deoxyribose backbone but lacking a base. Known analogs of natural nucleotides hybridize to nucleic acids in a manner similar to naturally occurring nucleotides, such as peptide nucleic acids (PNAs) and phosphorothioate DNA.

An “equivalent amino acid sequence” is a sequence that is not identical to the native amino acid sequence, but contains modifications, e.g., deletions, substitutions, inversions, insertion, and the like, that do not affect the biological activity of the protein as compared to the native protein. As used herein, the term “native” refers to proteins, peptides, nucleic acids, post-translational modifications, and the like that are intrinsic to the host cell and are not the result of recombinant techniques.

A “polypeptide” is a polymer comprising two or more amino acid residues (e.g., a peptide or a protein). The polymer can additionally comprise non-amino acid elements such as labels, quenchers, blocking groups, or the like and can optionally comprise modifications such as glycosylation or the like. The amino acid residues of the polypeptide can be natural or non-natural and can be unsubstituted, unmodified, substituted, or modified. An “amino acid sequence” is a polymer of amino acid residues (a protein, polypeptide, and the like) or a character string representing an amino acid polymer, depending on context.

Reference to sequence identity addresses the degree of similarity of two polymeric sequences, such as nucleic acid or protein sequences. Determination of sequence identity can be readily accomplished by persons of ordinary skill in the art using accepted algorithms and/or techniques. Sequence identity is typically determined by comparing two optimally aligned sequences over a comparison window, where the portion of the peptide or polynucleotide sequence in the comparison window can comprise additions or deletions (i.e., gaps) as compared to the reference sequence (which does not comprise additions or deletions) for optimal alignment of the two sequences. The percentage is calculated by determining the number of positions at which the identical amino acid residue or nucleic acid base occurs in both sequences to yield the number of matched positions, dividing the number of matched positions by the total number of positions in the window of comparison and multiplying the result by 100 to yield the percentage of sequence identity. Various software driven algorithms are readily available, such as BLAST N or BLAST P to perform such comparisons.

Disclosed are materials, compositions, and components that can be used for, can be used in conjunction with, can be used in preparation for, or are products of the disclosed methods and compositions. It is understood that, when combinations, subsets, interactions, groups, and the like, of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Disclosed herein are embodiments of a product and a method for assembling nanobodies (Nb) into polyvalent multimers to improve the effectiveness of Nb-based therapeutics and biotechnological tools.

Publications cited herein and the subject matter for which they are cited are hereby specifically incorporated by reference in their entireties.

### EXAMPLES

This Example describes that tetrazine-genetic code expansion enables designer multivalent nanobody assemblies.

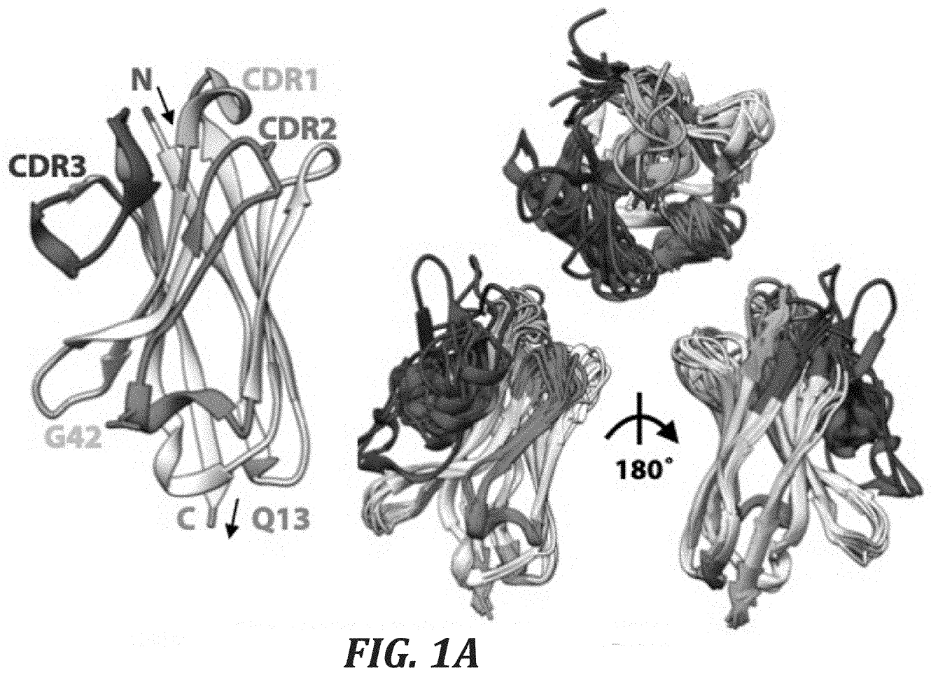

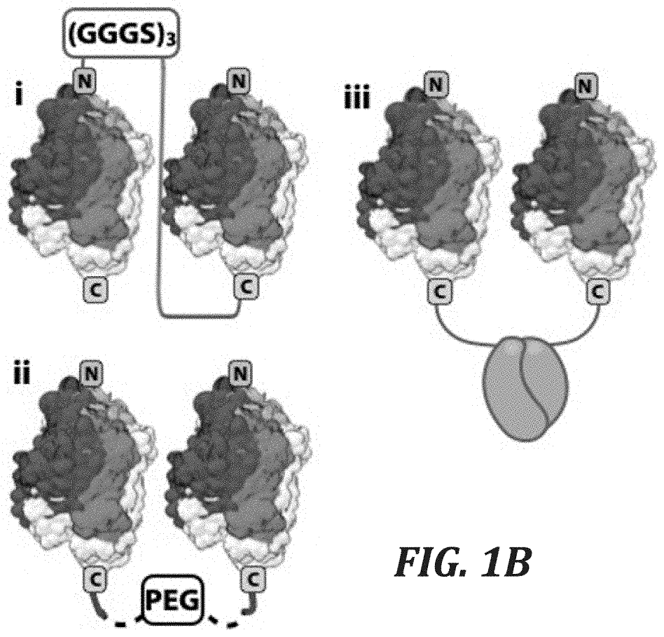

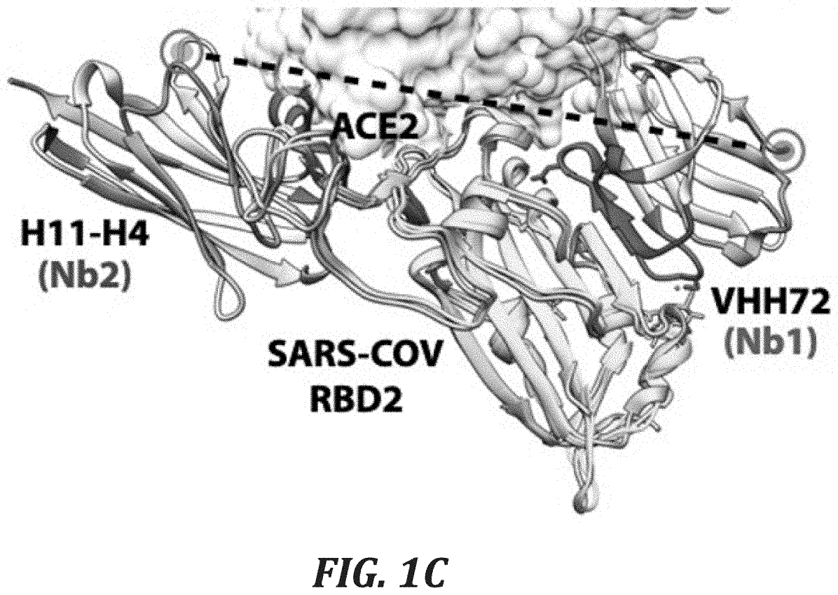

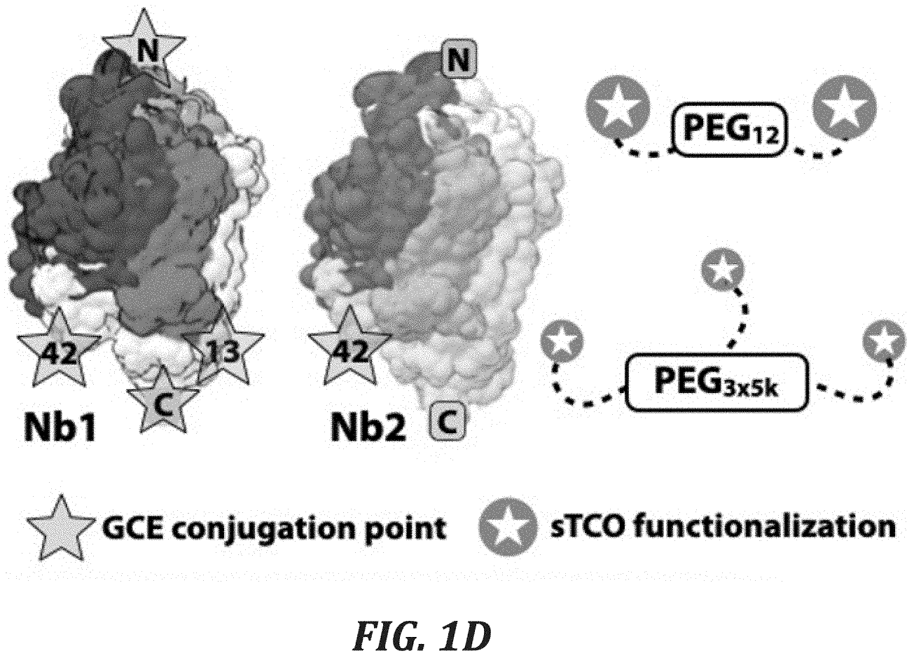

To assess the power of GCE-Tetrazine encoding as a platform for a versatile combinatorial approach to making defined high-affinity Nb assemblies in any system, two neutralizing Nbs were selected that recognized distinct epitopes on the RBD of the SARS-CoV-2 spike protein (FIG. 1C). For basic tests of the value of varying the point of linkage for Nb dimers, the “VHH72” Nb was selected—which is referred to as Nb1; it was one of the first examples of an Nb that bound SARS-CoV-2 RBD (D. Wrapp et al., Structural Basis for Potent Neutralization of Betacoronaviruses by Single-Domain Camelid Antibodies. Cell, 181(5):1004-1015, (2020)). Although originally selected to bind SARS-CoV-1 RBD, Nb1 exhibited modest binding against SARS-CoV-2 RBD, and dimerization improved both binding and neutralization abilities.

The forms of Nb1 created for this study include a genetically-linked dimer control (referred to as Nb1-Nb1) and monomers with the Tet3.0 ncAA installed at one of four distinct GCE-enabled linkage points—at the N- and C-termini (referred to as Nb1_{N} and Nb1_{C} respectively) and at the framework positions Gln13 (Nb1₁₃) and Gy42 (Nb1₄₂), two structurally distant Nb framework sites shown to be amenable to ncAA introduction without compromising stability or binding (K. W. Yong, D. Yuen, M. Z. Chen, A. P. R. Johnston, Engineering the Orientation, Density, and Flexibility of Single-Domain Antibodies on Nanoparticles To Improve Cell Targeting. ACS Appl. Mater. Interfaces, 12:5593-5600 (2020)) (FIG. 1D). The Nb1 interactions with SARS-CoV-2 RBD chiefly occur along the side of the Nb, extending down almost its entire length (FIG. 1C), and none of the four sites should directly interfere with the binding region (FIG. 1A).

The second Nb—designated Nb2—is the “H11-H4” Nb (J. Huo et al., Neutralizing nanobodies bind SARS-CoV-2 spike RBD and block interaction with ACE2. Nat. Struct. Mol. Biol., 27:846-854 (2020)). It recognizes a distinct RBD epitope compared with Nb1 (FIG. 1C) and so can be used for making biparatopic heterodimers. Nb2 boasts a low nanomolar K_{D} (5 nM) as a monomer and exhibits potent neutralization ability as an Fc mediated dimer (ND₅₀ 4 to 6 nM) (FIG. 1B). For the linkage chemistry, the tetrazine 3.0 (Tet3.0) ncAA was used, which is compatible with E. coli and mammalian GCE systems, (H. S. Jang, S. Jana, R. J. Blizzard, J. C. Meeuwsen, R. A. Mehl, Access to Faster Eukaryotic Cell Labeling with Encoded Tetrazine Amino Acids J. Am. Chem. Soc., 142:7245-7249 (2020)) along with 2-headed (PEG)₁₂ linkers or a 3-headed (PEG)₅ₖ linker with reactive sTCO head groups (FIG. 1D).

Producing Ligation-Capable Nb Building Blocks

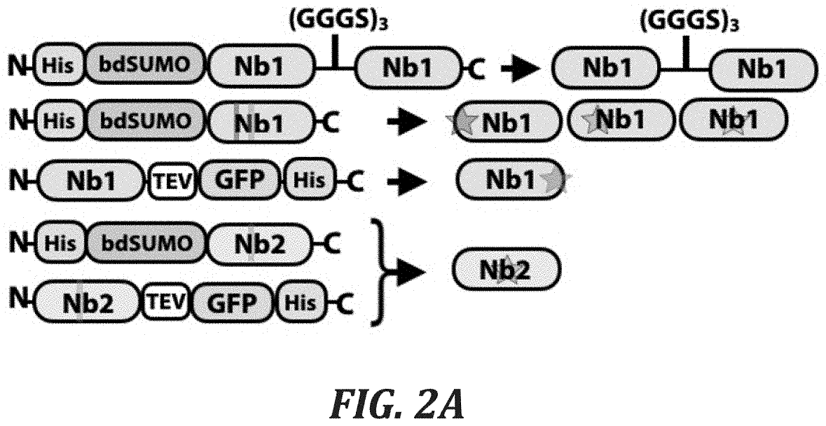

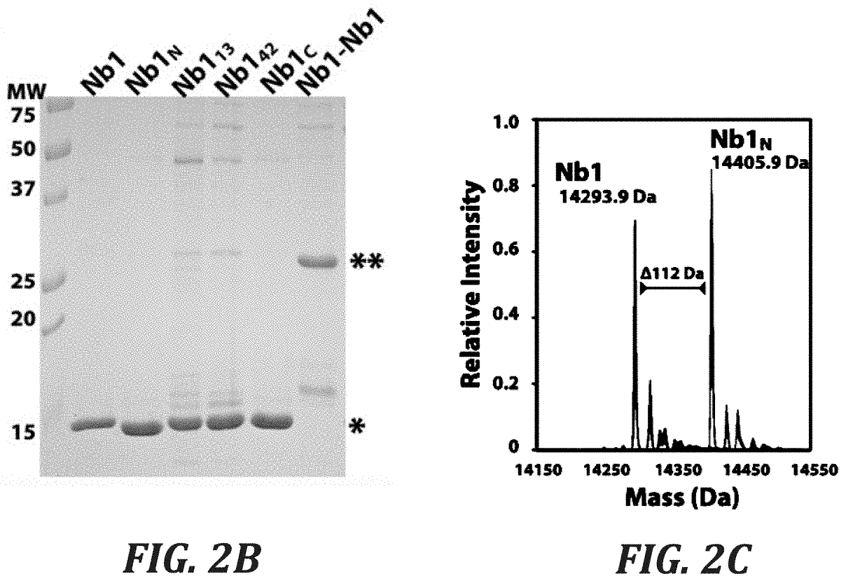

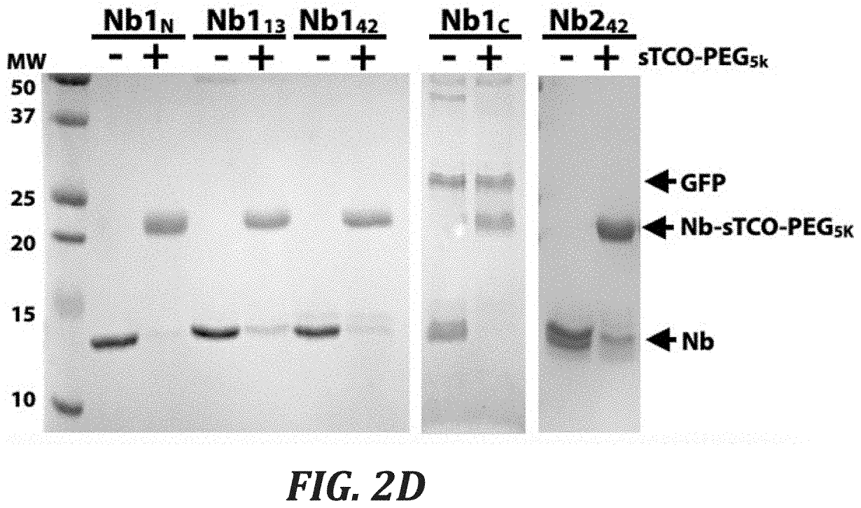

Most constructs were designed with cleavable N-terminal His- and bdSUMO-tags to facilitate expression and purification (FIG. 2A). This included the control “tail to head” genetically linked dimer of Nb1 (Nb1-Nb1) that had a (GGGS)₃ (SEQ ID NO:27) linker as described by Wrapp et al., (D. Wrapp et al., Structural Basis for Potent Neutralization of Betacoronaviruses by Single-Domain Camelid Antibodies. Cell, 181(5):1004-1015 (2020); D. Wrapp et al., Structural Basis for Potent Neutralization of Betacoronaviruses by Single-Domain Antibodies Cell, 181(6):1436-1441 (2020)). One exception was the Nb1 construct encoding Tet3.0 at the C-terminus for which truncation products not incorporating Tet3.0 would be nearly identical in length and difficult to purify away from the desired full-length product. To avoid this potential problem, a TEV-cleavable GFP domain and His tag was appended to the C-terminus of the construct. In some embodiments, this C-terminal extension for the Nb2 construct was used in the assembly of a Nb1-Nb2 heterodimer, so that the additional mass of the GFP and its fluorescence would aid isolation of the desired heterodimer product from potentially contaminating homodimer products.

Expression and purification, including cleavage of the solubility tags, for all targeted Nb1 and Nb2 forms resulted in reasonable to excellent yields of tet-encoded Nb, ranging from 5 to 30 mg/L of culture (FIG. 2B and Table 1).

TABLE 1
Expression yields for Nbs after purification.

| Construct | Expression yields (mg/L culture) |
|---|---|
| Nb1 WT | 15 |
| Nb1 − Nb1 | 5.0 |
| Nb1 − Nb1 − Nb1 | 0.6 |
| Nb1_{N} | 25 |
| Nb1₁₃ | 4.0 |
| Nb1₄₂ | 6.0 |
| Nb1_{C} | 10 |
| Nb2₄₂ | 35 |

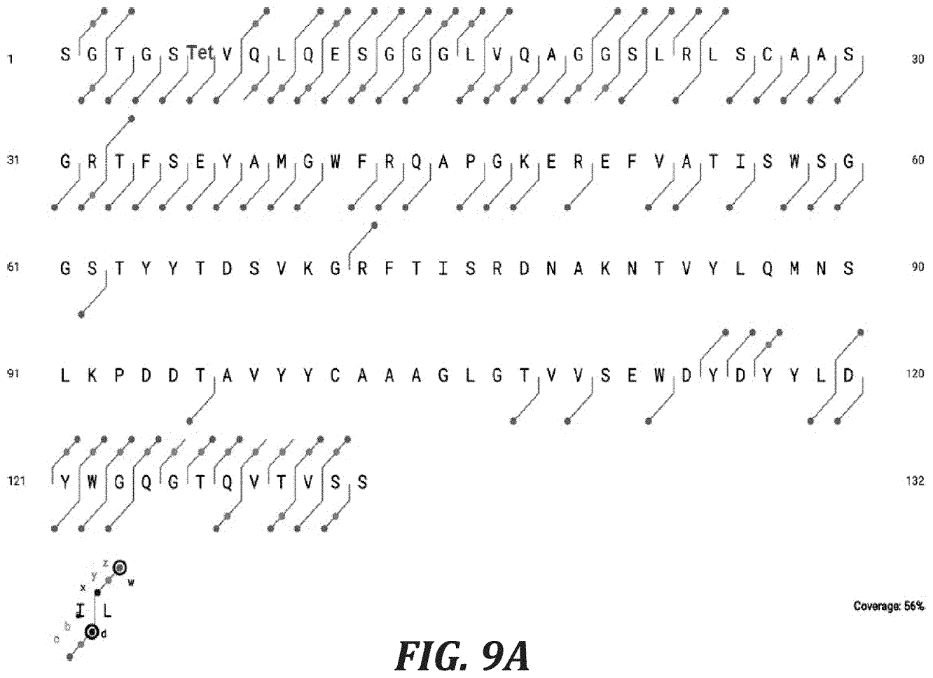

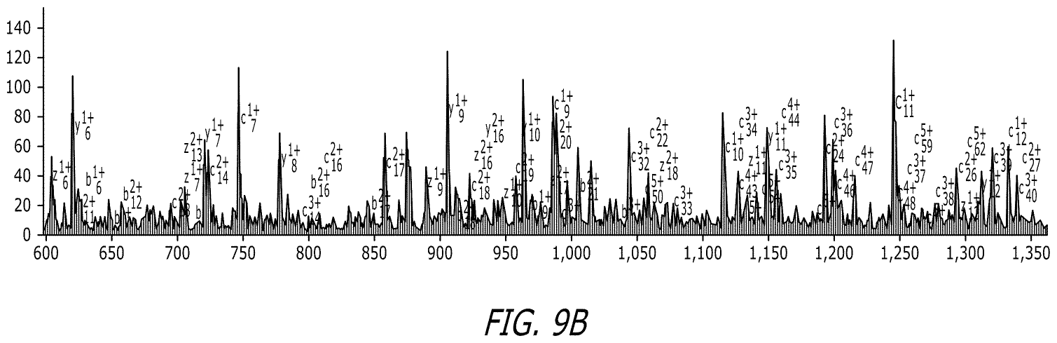

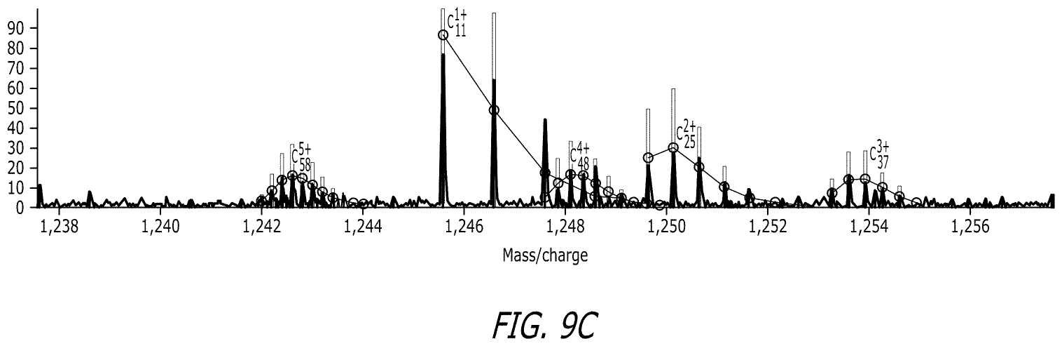

The incorporation of intact Tet3.0 was confirmed using Nb1N as a representative example, with electrospray ionization mass spectrometry (ESI MS), with a mass shift of +112 Da observed in Nb1N compared to wild type (WT) agreeing well with the expected +113 Da difference between Tet3.0 and the native Gln (FIG. 2C). The precise location of the Tet3.0 was additionally confirmed with mass spectrometry confirming the site-specificity of our technique (FIG. 9). The reactivity of each Tet3.0-Nb was then tested by incubating it with an sTCO-PEG5000 polymer. In every case, over 95% of the protein sample showed a mobility shift in SDS-PAGE, indicating excellent reactivity of the incorporated Tet3.0 (FIG. 2D).

The Site of Homodimerization of Nb1 Impacts Binding Ability

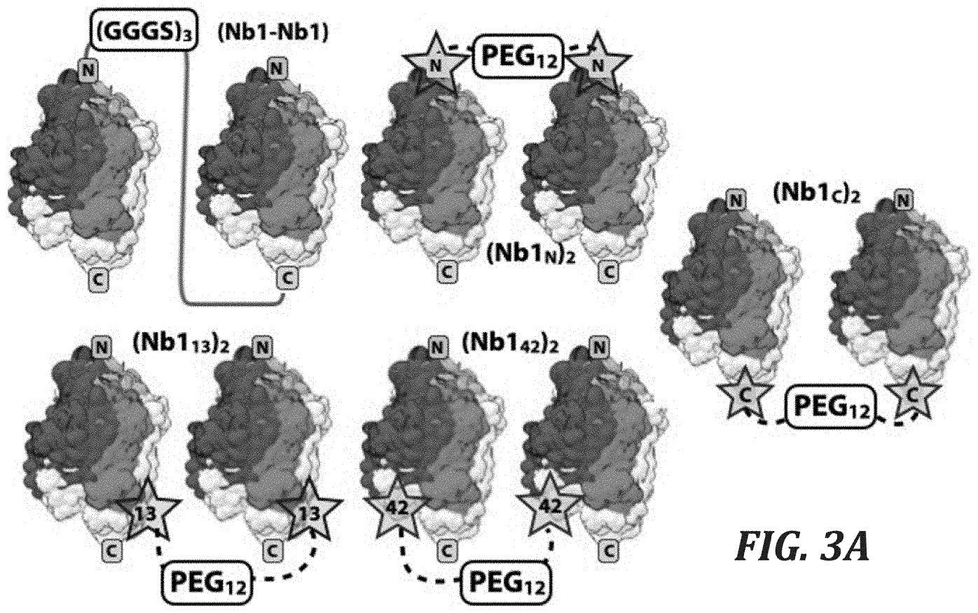

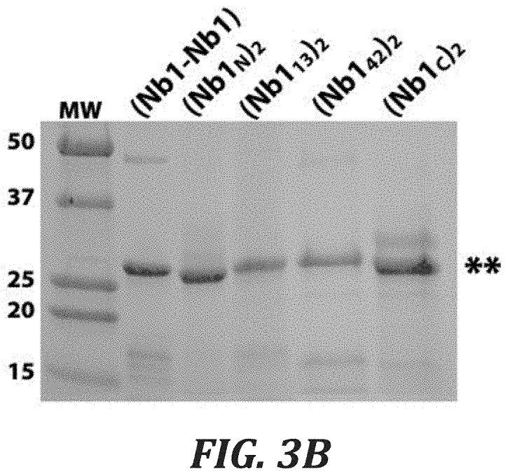

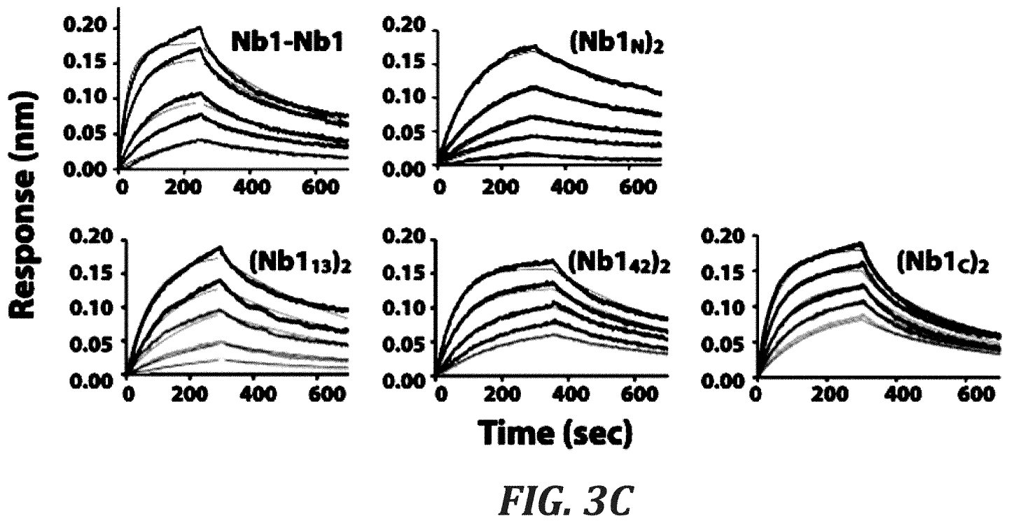

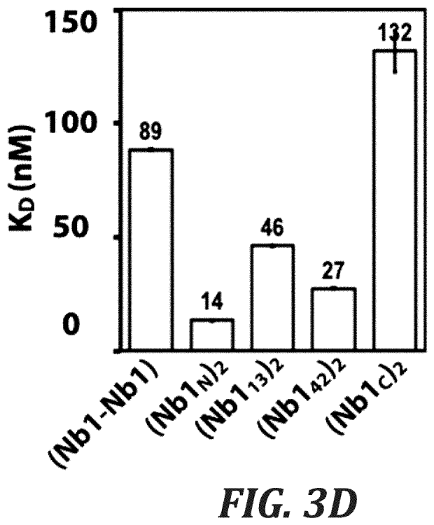

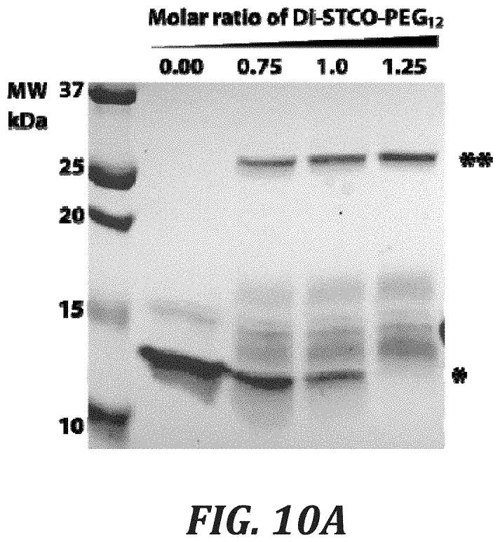

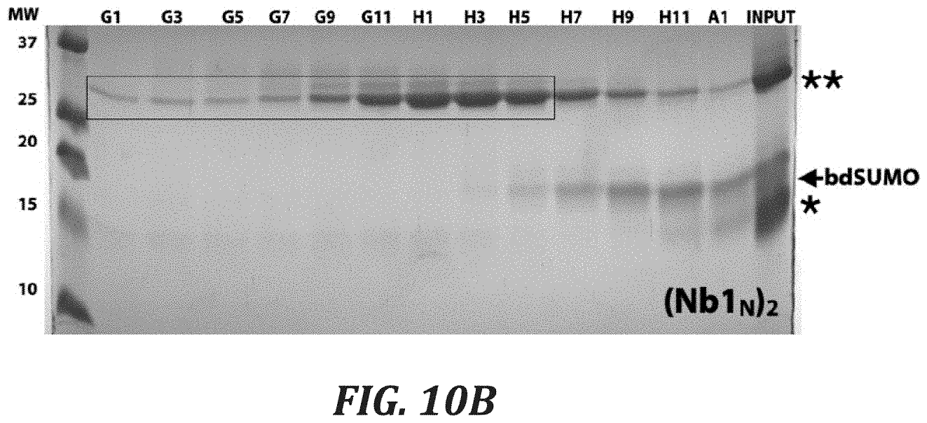

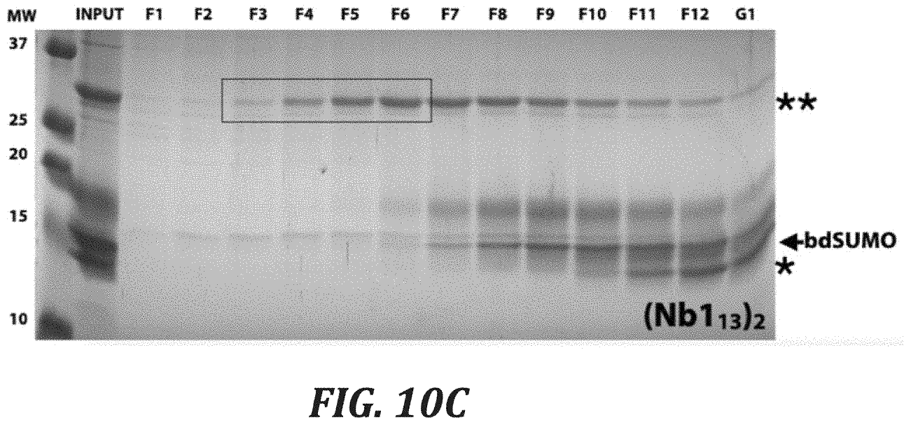

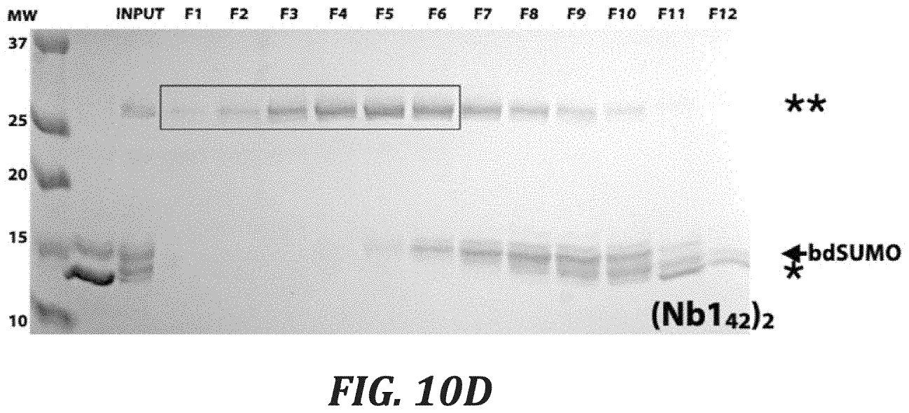

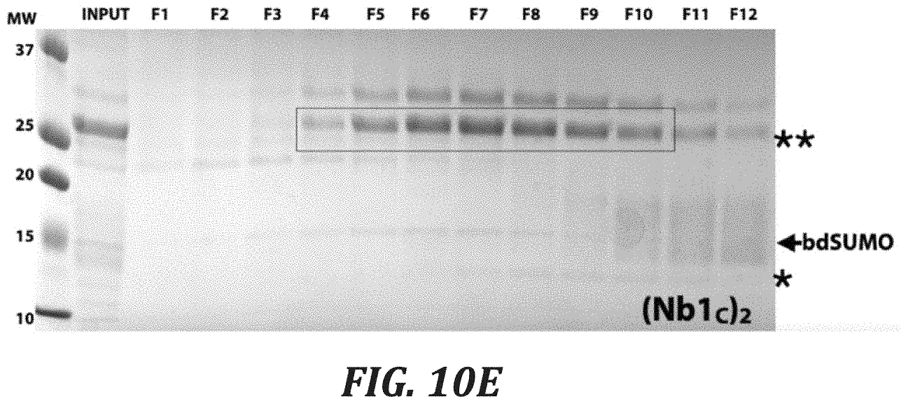

In some embodiments, chemically conjugated homodimers were generated of each Nb1 form: Nb1_{N}, Nb1₁₃, Nb1₄₂, Nb1_{C} and the Nb1-Nb1 control (FIG. 3A). The goal at this stage was only to make sufficient material for testing, a simple one-pot reaction was attempted and found to be adequate (see methods). Each Tet3.0-Nb1 form was reacted with the 2-headed sTCO (PEG)₁₂ linker (FIG. 1D), with a brief 10-minute incubation on ice yielding 40 to 80% homodimer product that was purified away from the unreacted material with size exclusion chromatography (SEC) (FIG. 3B and FIG. 10). With biolayer interferometry (BLI), the affinity of each homodimer was evaluated for immobilized SARS-CoV-2 RBD, and in every case the data fit reasonably to a single binding event (FIG. 3C and Table 2). The control Nb1-Nb1 genetic “tail to head” dimer yielded K_{D} of ˜88 nM, providing a reference value to which the others could be compared (FIG. 3D). (Nb1₄₂)₂, (Nb1₁₃)₂ and (Nb1_{N})₂ dimers bound 3- to 7-fold tighter and (Nb1_{C})₂ bound ˜1.5-fold weaker. The higher affinities of (Nb1₄₂)₂ and (Nb1_{N})₂ were due to both a higher association rate and a slower dissociation rate whereas the (Nb1₁₃)₂ improvement was due solely to a slower disassociation rate (Table 2).

TABLE 2
Binding kinetics of the Nb assemblies for the SARS-CoV-2 RBD.

| Construct | kₒₙ (1/Ms) × 10⁴ | k_{off} (1/s) × 10⁻³ | K_{d} (nM) |
|---|---|---|---|
| Nb1 − Nb1 | 3.54 ± 0.04 | 3.14 ± 0.03 | 89 ± 1 |
| (Nb1_{N})₂ | 8.58 ± 0.04 | 1.186 ± 0.0006 | 13.8 ± 0.1 |
| (Nb1₁₃)₂ | 3.38 ± 0.07 | 1.56 ± 0.03 | 46 ± 1 |
| (Nb1₄₂)₂ | 7.31 ± 0.04 | 2.01 ± 0.01 | 27.5 ± 0.2 |
| (Nb1_{C})₂ | 4.81 ± 0.01 | 6.4 ± 0.4 | 132 ± 8 |
| (Nb1_{N}) − (Nb2₄₂) | 15.07 ± 0.08 | 0.593 ± 0.003 | 3.93 ± 0.03 |
| Nb1 − Nb1 − Nb1 | 5.79 ± 0.08 | 1.56 ± 0.04 | 27.9 ± 0.8 |
| (Nb1_{N})₃ | 24.6 ± 0.2 | 0.566 ± 0.004 | 2.31 ± 0.02 |

Heterodimeric and Trimeric Nbs with Potential as Enhanced SARS-CoV-2 Therapeutics

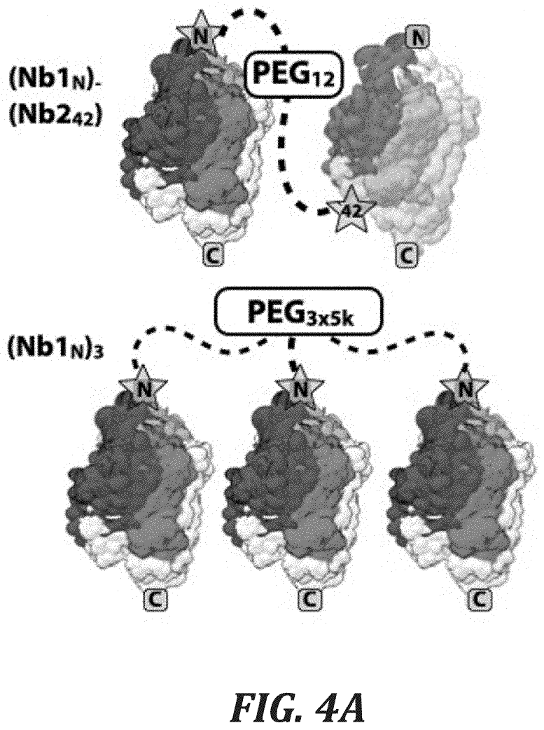

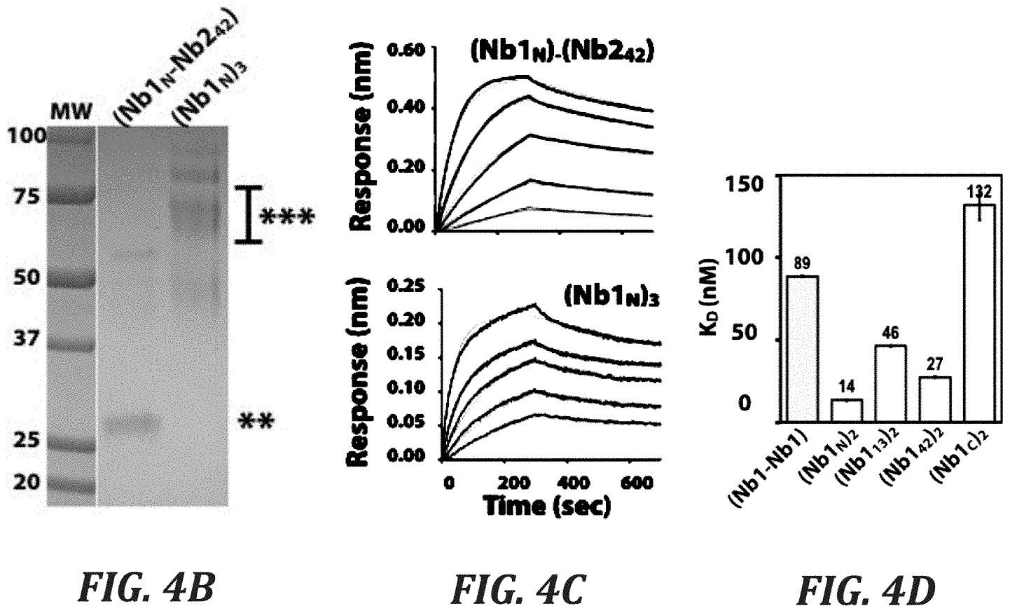

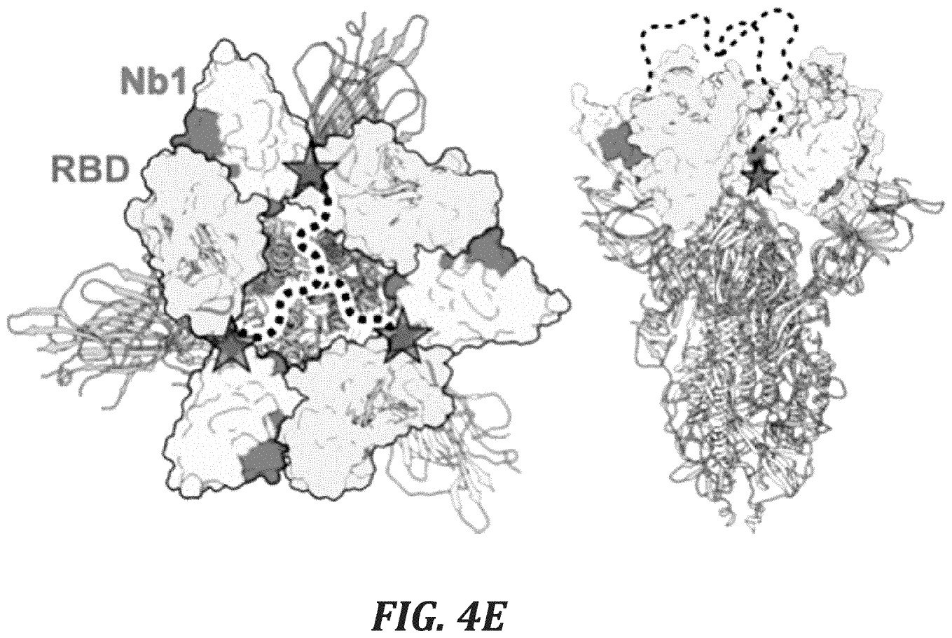

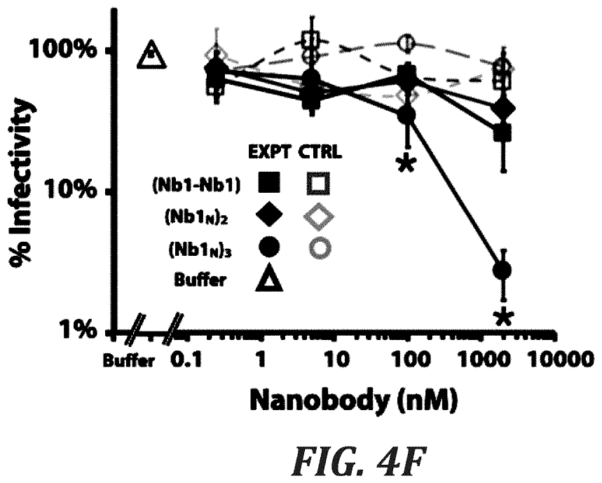

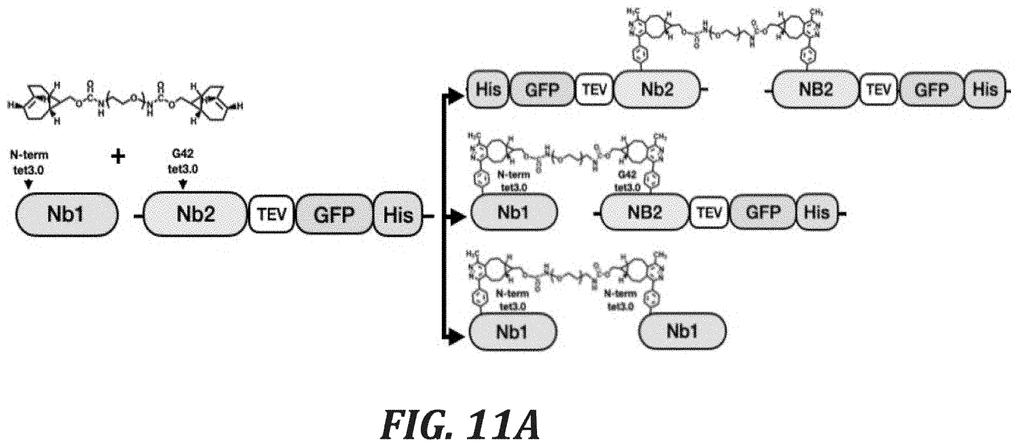

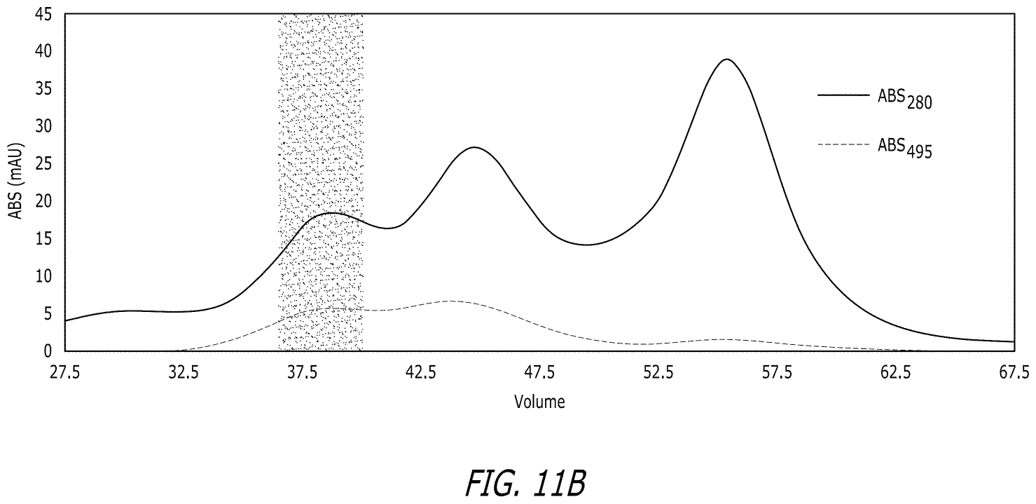

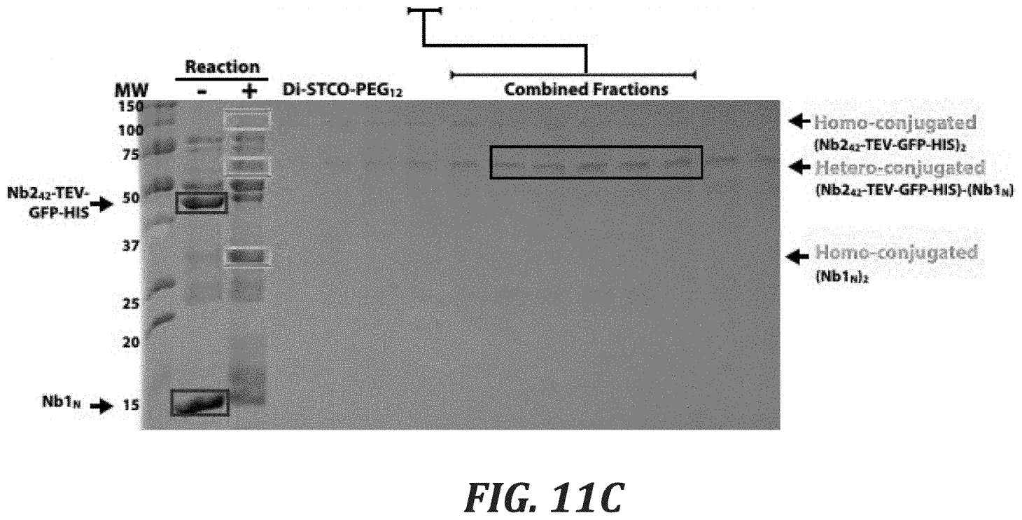

Of great interest was to show this technology could also be used to make a heterodimeric Nb assembly targeting two independent epitopes on the RBD, which should enhance viral neutralization and hinder the development of viral resistance (P. A. Koenig et al., Structure-guided multivalent nanobodies block SARS-CoV-2 infection and suppress mutational escape. Science, 371(6539):eabe6230 (2021)). To synthesize a GCE-enabled biparatopic Nb dimer, Nb1_{N} (as the tightest binder among the Nb1 constructs) was selected and it was coupled with Nb2 (introduced above; FIG. 1C). For Nb2, Gly42 was selected as a linkage site that should not interfere with its RBD interactions and that is on the side of Nb2 expected to be closest to Nb1 when both are bound to a single RBD (FIG. 1C and FIG. 4A). To facilitate resolution of the heterodimeric product from a potential mixture of reaction products, a cleavable GFP tag was appended to Nb2, adding both bulk and a fluorescent signature to the Nb1_{N}-Nb2₄₂ heterodimer compared to the (Nb1_{N})₂ homodimer. The un-cleaved Nb2₄₂-TEV-GFP protein (FIG. 2A) and an excess of Nb1_{N} was reacted with the 2-headed sTCO (PEG)₁₂ linker (FIG. 1D) in a one pot manner, and after purification (FIG. 11 and FIG. 4B), the Nb1_{N}-Nb2₄₂ heterodimer analyzed by BLI yielded a K_{D} of ˜4 nM for 1:1 binding of the RBD (FIG. 4C, 4D and Table 2).

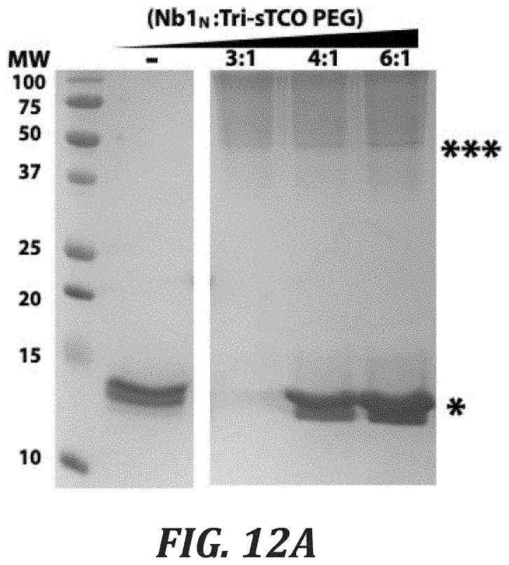

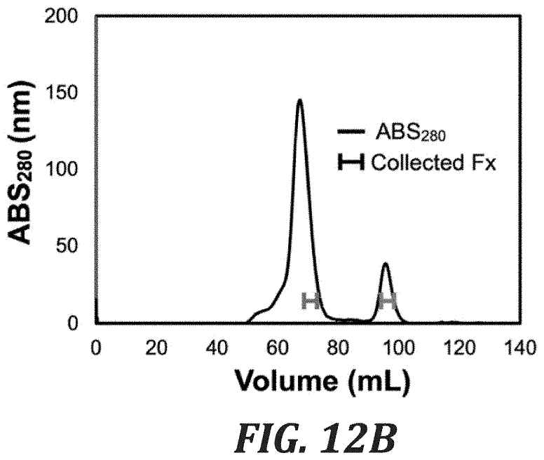

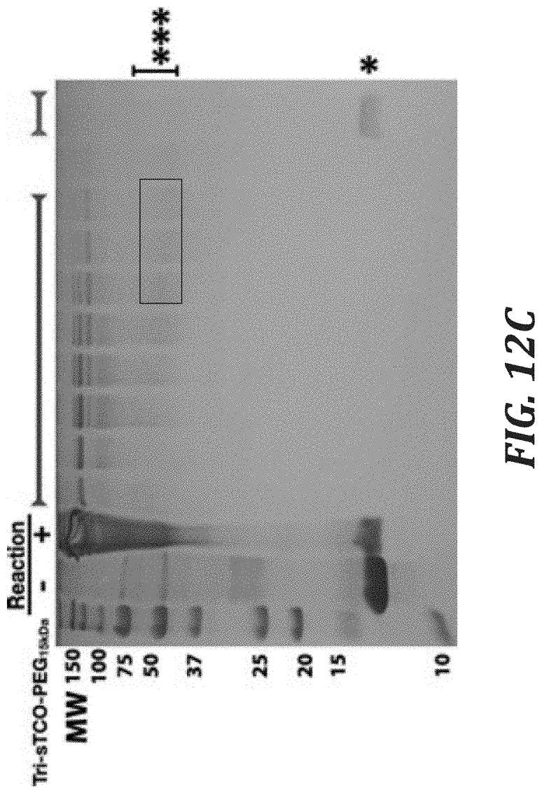

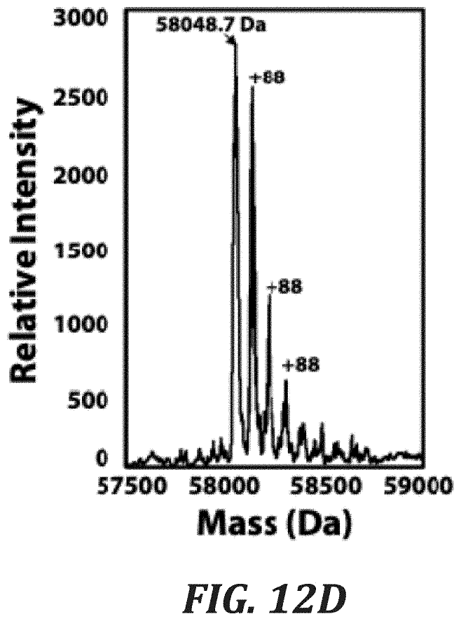

To highlight the potential of the GCE-conjugation strategy to make Nb-assemblies matching specific aspects of target topologies, an Nb1 trimer was assembled to match the stoichiometry of the trimeric SARS-CoV-2 spike protein (FIG. 4A). Using Nb1_{N}, the tightest binding of the Nb1 constructs, a trimer was generated by conjugating it with a 3-armed Tri-sTCO PEG 5K linker (FIG. 1D) in a simple, one pot reaction, with excess Tet3.0-Nb1_{N} to ensure full loading of each tri-sTCO linker (FIG. 12A). The fully loaded Nb-trimer, (Nb1_{N})₃, was separated from other products (FIG. 12B) and mass spectrometry showed it to be a single population with a mass of ˜58 kDa (FIG. 4C). By BLI, (Nb1_{N})₃ exhibited a K_{D} of ˜2 nM for the SARS-CoV-2 RBD, roughly 6-fold tighter than the (Nb1_{N})₂ dimer stemming from an ˜3-fold larger kₒₙ and ˜2-fold smaller k_{off} and 44-fold tighter than the genetic dimer benchmark (FIG. 4C, FIG. 4D, and Table 2).

GCE-Enabled Nb Assemblies Neutralize SARS-CoV-2 Pseudotyped Viruses

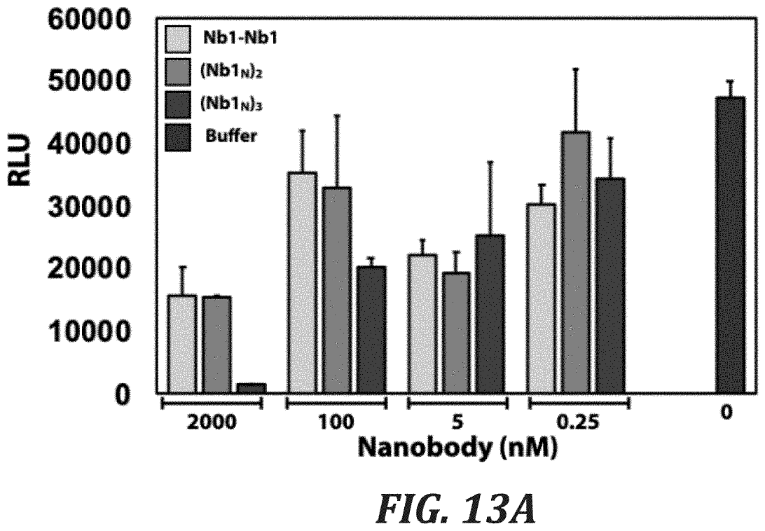

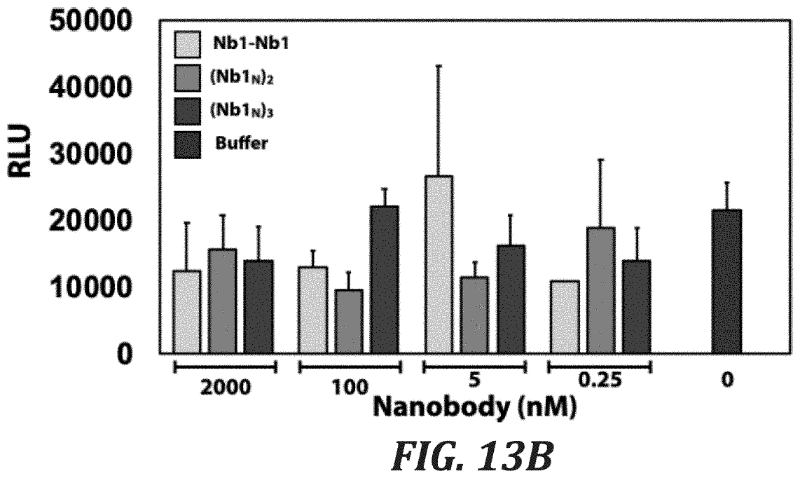

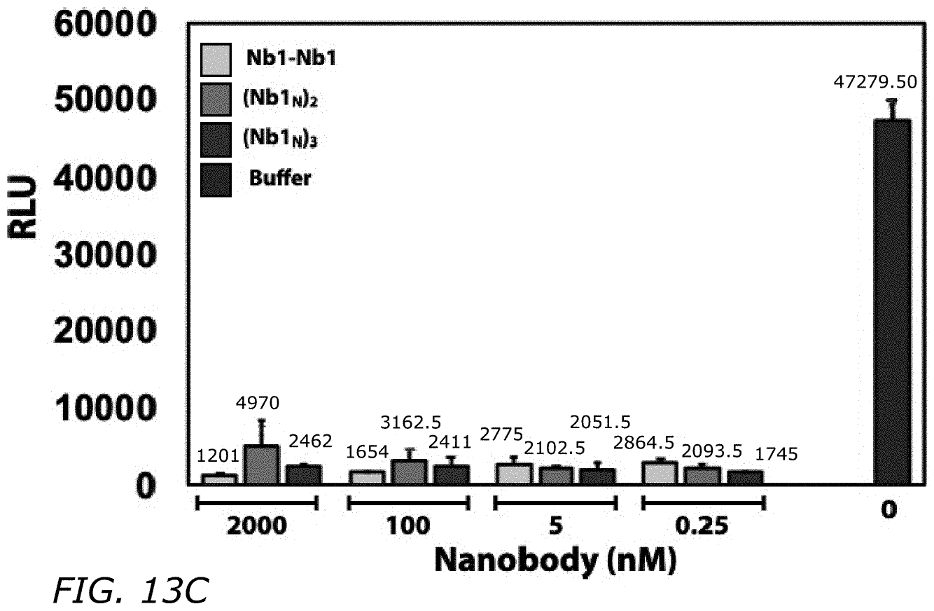

It is important to note that a monomeric form of the RBD was immobilized for the BLI assays, so the true effects of Nb trimerization against a trimeric S-protein target may differ from what is seen in this example (FIG. 4A). To directly test the extent to which the improved binding of (Nb_{N})₃ seen in the BLI experiments translates into an increased SARS-CoV-2 neutralization ability, (Nb_{N})₃ was compared with (Nb_{N})₂ and Nb1-Nb1 in a standard luciferase-based pseudovirus neutralization assay with HIV-derived lentiviral particles containing the S protein of SARS-CoV-2 (FIG. 4A) (K. H. D. Crawford et al., Protocol and Reagents for Pseudotyping Lentiviral Particles with SARS-CoV-2 Spike Protein for Neutralization Assays. Viruses, 12(5):513 (2020); J. Kim, A. Mukherjee, D. Nelson, A. Jozic, G. Sahay, Rapid generation of circulating and mucosal decoy ACE2 using mRNA nanotherapeutics for the potential treatment of SARS-CoV-2. bioRxiv, (2020)). Additionally, the neutralization ability of the same set of Nbs was evaluated on pseudovirus not decorated with SARS-CoV-2 spike protein that should maintain infectivity regardless of Nb pre-incubation (VSG control, FIG. 13B). While all three Nb assemblies showed some neutralization, the (Nb_{N})₃-trimer was significantly more effective than either of the dimers, achieving 96% neutralization at the highest concentration tested (FIG. 4E), and was the only nanobody conjugate to exhibit significant neutralization ability over the VSG control. Between the two dimers, (Nb_{N})₂ and (Nb1-Nb1), there was no significant difference in neutralization efficacy.

Engineered proteins have transformed the therapeutic landscape, providing powerful new approaches to treat disease with improved efficacy (L. Presta, Antibody engineering for therapeutics. Curr. Opin. Struct. Biol., 13:519-525, (2003)), as seen by the success of many drug-antibody conjugates, bi-specific antibodies and imaging agents. As the field begins to include Nbs and transitions to developing therapeutic protein-protein conjugates that are tailored to targets with unique and dynamic topologies, it is essential to develop efficient and modular ways to connect small and sensitive therapeutic proteins in a manner that enhances their ability and utility as therapeutics.

Using two previously characterized Nbs against distinct epitopes on the SARS-CoV-2 spike protein RBD as practical examples, this Example describes that GCE-Tetrazine encoding technology provides a flexible and facile, generally applicable approach for generating diverse Nb assemblies with unique conjugation points. This Example evaluated (Nb1_{N})₂, (Nb1_{C})₂ termini homodimeric conjugations along with (Nb1₁₃)₂, (Nb1₄₂)₂ framework homodimeric conjugations in direct comparison to a (Nb1-Nb1) genetic conjugation. An unexpected finding is that Nb1_{N} conjugation led to the tightest binding even with the close proximity of the N-terminus to the CDR regions, highlighting the general need for screening multiple conjugation orientations. While not wishing to be bound by any particular mechanism, this result was possible because the binding interface, comprised of both CDR2 and CDR3 loops, extends down the side of the Nb instead of being centered at its top. This small set of tetrazine-linked Nb assemblies shows not only that the approach is viable, but also demonstrates that the site of attachment is a crucial design parameter that must be leveraged to access the Nb assemblies with improved properties.

The high efficiency of GCE-tetrazine mediated conjugation removed the restriction of homodimeric conjugations, as this Example shows that heterodimerization and trimerization are easily achievable. These conjugation options are specifically advantageous for the development of SARS-CoV-2 therapeutics since they precisely target the trimeric organization of the spike protein and targeting independent RBD epitopes to limit viral escape (FIG. 4A) (P. A. Koenig et al., Structure-guided multivalent nanobodies block SARS-CoV-2 infection and suppress mutational escape. Science, 37(6530):eabe6230. doi: 10.1126/science.abe6230. Epub 2021 Jan. 12; F. Chen, Z. Liu, F. Jiang, Prospects of Neutralizing Nanobodies Against SARS-CoV-2. Front. Immunol., 12:690742 (2021)). In some embodiments, trimerization of Nbs, utilizing an ideal binding locus, also resulted in a greatly enhanced binding ability. In addition to matching the trimeric spike protein organization, improved binding can be attributed to avidity-increasing effects of multimerization and optimized conjugation sites. Importantly, this increased trimerized Nb binding ability is significant enough to translate into increased neutralization ability, as determined by our SARS-CoV-2 pseudovirus assay results. This result agrees with other studies, which have found that flexibility in protein-multimer engineering translates into improved ability (A. C. Hunt et al., Multivalent designed proteins protect against SARS-CoV-2 variants of concern. bioRxiv, 2021 Jul. 7; 2021.07.07.451375.doi: 10.1101/2021.07.07.451375. Preprint (2021)).

The disclosed GCE-Tetrazine encoding technology provides both optimal chemistry and unfettered attachment points to functionalize Nbs with no restrictions from bioorthogonal conjugation conditions. Furthermore, the recent optimization of Tet3.0 GCE technology in mammalian cells (H. S. Jang, S. Jana, R. J. Blizzard, J. C. Meeuwsen, R. A. Mehl, Access to Faster Eukaryotic Cell Labeling with Encoded Tetrazine Amino Acids. J. Am. Chem. Soc., 142:7245-7249 (2020)) and two step, 80% yield chemical synthesis of Tet3.0 ncAA, allows for the production of Tet-Nb based therapeutics from conventional mammalian sources. Thus, this conjugation approach allows access to the full combinatorial diversity of Nb assemblies with facile modulation of the sites of conjugation on Nbs, the linker lengths and geometries and the types of Nb used. This Example demonstrates how the rapid screening of these variables is critical when maximizing binding of different fused Nb architectures. This rapid assembly and screening of combinatorial fused Nb architectures will advantage therapeutic Nb development (D. De Vlieger, M. Ballegeer, I. Rossey, B. Schepens, X. Saelens, Single-Domain Antibodies and Their Formatting to Combat Viral Infections. Antibodies (Basel); 8(1):1.doi: 10.3390/antib8010001 (2018)) when optimizing their therapeutic window, lifetime, toxicity, and the like, and will provide a unique advantage when combating rapidly evolving viral targets, such as SARS-CoV-2.

Molecular cloning of plasmids used in this study (Table 3) were constructed in the following manner

TABLE 3
Plasmids used in the study.

| | | | | | Size | | | |
|---|---|---|---|---|---|---|---|---|
| No. | Plasmid name | Promoter(s) | Ori | Res. | (bp) | Primers | Template | Backbone |
| 1 | pDULE2-Tet3.0 | lpp(aaRS) | p15 | Spec | | — | — | — |
| | | lpp(tRNA) | | | | | | |
| 2 | pBAD-His-bdSUMONb1(A) | araBAD | pBR322 | AMP | 4691 | 1-4 | — | pBAD |
| | | | | | | | | (NcoI/XhoI) |
| 3 | pBAD-His- | araBAD | pBR322 | AMP | 5140 | 1-4 | | pBAD |
| | bdSUMONb1(A)- | | | | | | | (NcoI/XhoI) |
| | (GGGGS)3- Nb1(B) | | | | | | | |
| 4 | pBAD-His- | araBAD | pBR322 | AMP | 4691 | 1, 4-6 | 1 | pBAD |
| | bdSUMONb1(A)[Q1TAG] | | | | | | | (NcoI/XhoI) |
| 5 | pBAD-His- | araBAD | pBR322 | AMP | 4691 | 1, 4, 7-8 | 1 | pBAD |
| | bdSUMONb1(A)[Q13TAG] | | | | | | | (NcoI/XhoI) |
| 6 | pBAD-His- | araBAD | pBR322 | AMP | 4691 | 1, 4, 9-10 | 1 | pBAD |
| | bdSUMONb1(A)[G42TAG] | | | | | | | (NcoI/XhoI) |
| 7 | pBADNb1(A)[G129TAG]- | araBAD | pBR322 | AMP | 5157 | 4, 11-13 | 1 | pBAD |
| | TEV-GFP-HIS | | | | | | | (NcoI/XhoI) |
| 8 | pBAD-His-bdSUMONb2 | araBAD | pBR322 | AMP | 4694 | 1, 4, 14-15 | — | pBAD |
| | | | | | | | | (NcoI/XhoI) |
| 9 | pBAD-His- | araBAD | pBR322 | AMP | 4694 | 1, 4, 16-17 | 8 | pBAD |
| | bdSUMONb2[G42TAG] | | | | | | | (NcoI/XhoI) |
| 10 | pBADNb2[G42TAG]- | araBAD | pBR322 | AMP | 5129 | 4, 18-20 | 8 | pBAD |
| | TEVGFP-His | | | | | | | (Ncol/XhoI) |

Previously published nanobody sequences (VHH72 (D. Wrapp et al., Structural Basis for Potent Neutralization of Betacoronaviruses by Single-Domain Camelid Antibodies. Cell, 2020 May 28; 181(5):1004-1015.e15.doi: 10.1016/j.cell.2020.04.031. Epub 2020 May 5.), H11H4 (J. Huo et al., Neutralizing nanobodies bind SARS-CoV-2 spike RBD and block interaction with ACE2. Nat. Struct. Mol. Biol., 27:846-854 (2020)), containing at least 25 bp of homology at their flanking ends to either the vector backbone or other fragments, were optimized for expression in E. coli and synthesized by Integrated DNA Technologies (Coralville, IA) (Table 4).

TABLE 4
Genes used in this study. Codons that are mutated to TAG sited are
underlined.

| Gene | Sequence | SEQ ID NO: |
|---|---|---|
| His- | AGCGCCGCCGGTGGTGAAGAAGATAAAAAACCGGCGGGCGGCGAAGGCGGTGG | 1 |
| bdSUMO | CGCGCATATCAACCTGAAAGTGAAAGGTCAGGATGGTAACGAAGTGTTCTTCCGC | |
| | ATTAAACGCAGCACCCAGCTGAAGAAACTGATGAACGCCTATTGCGATCGCCAGA | |
| | GCGTCGATATGACCGCCATTGCCTTCCTGTTTGATGGCCGCCGTCTGCGTGCGGAACA | |
| | GACGCCGGACGAACTGGAAATGGAAGACGGCGATGAAATTGATGCGATGCTGCA | |
| | TCAGACCGGCGG | |
| sfGFP | GTTAGCAAAGGTGAAGAACTGTTTACCGGCGTTGTGCCGATTCTGGTGGAACTGG | 2 |
| | ATGGTGATGTGAATGGCCATAAATTTAGCGTTCGTGGCGAAGGCGAAGGTGATGC | |
| | GACCAACGGTAAACTGACCCTGAAATTTATTTGCACCACCGGTAAACTGCCGGTT | |
| | CCGTGGCCGACCCTGGTGACCACCCTGACCTATGGCGTTCAGTGCTTTAGCCGCT | |
| | ATCCGGATCATATGAAACGCCATGATTTCTTTAAAAGCGCGATGCCGGAAGGCTA | |
| | TGTGCAGGAACGTACCATTAGCTTCAAAGATGATGGCACCTATAAAACCCGTGCG | |
| | ATTTTAAAGAAGATGGCAACATTCTGGGTCATAAACTGGAATATAATTTCAACAG | |
| | CCATAATGTGTATATTACCGCCGATAAACAGAAAAATGGCATCAAAGCGAACTTTAA | |
| | AATCCGTCACAACGTGGAAGATGGTAGCGTGCAGCTGGCGGATCATTATCAGCAGA | |
| | ATACCCCGATTGGTGATGGCCCGGTGCTGCTGCCGGATAATCATTATCTGAGCAC | |
| | CCAGAGCGTTCTGAGCAAAGATCCGAATGAAAAACGTGATCATATGGTGCTGCTG | |
| | GAATTTGTTACCGCCGCGGGCATTACCCACGGTATGGATGAACTGTATAAA | |
| Nb1A | CAAGTGCAGTTGCAAGAATCTGGTGGCGGATTGGTCCAAGCGGGGGGTTCGCTTC | 3 |
| (VHH72) | GAAGTTAAATTTGAAGGCGATACCCTGGTGAACCGCATTGAACTGAAAGGTATTG | |
| | GCCTGAGTTGTGCCGCGTCTGGACGCACTTTTTCTGAGTACGCAATGGGCTGGTTC | |
| | CGCCAGGCTCCGGGCAAAGAACGTGAATTCGTTGCGACCATTTCCTGGAGCGGAG | |
| | GCTCTACGTACTACACCGATTCGGTTAAGGGCCGTTTCACCATTTCTCGTGACAATGC | |
| | GAAGAATACGGTTTATCTTCAAATGAACTCATTGAAGCCAGACGATACAGCGGTC | |
| | TATTACTGTGCTGCAGCGGGATTAGGTACGGTAGTGTCGGAATGGGATTATGATT | |
| | ATTATTTAGATTATTGGGGCCAGGGCACGCAGGTAACAGTTAGTTCA | |
| Nb1B | TCTCAAGTTCAATTACAGGAGTCCGGGGGAGGCCTTGTGCAAGCTGGTGGTAGTT | 4 |
| (VHH72) | TACGCCTGTCTTGCGCAGCGTCTGGGCGCACCTTCTCCGAATACGCAATGGGTTG | |
| | GTTCCGTCAAGCACCTGGAAAGGAGCGCGAGTTTGTTGCTACTATTTCCTGGTCCG | |
| | GCGGTAGTACTTACTATACGGACAGCGTCAAGGGCCGCTTTACGATTTCGCGTGACAA | |
| | CGCTAAAAACACCGTGTATTTACAGATGAACTCATTAAAGCCCGACGATACGGCC | |
| | GTGTATTATTGTGCCGCCGCAGGCTTAGGTACAGTTGTTTCAGAATGGGATTACGAC | |
| | TATTACTTAGATTATTGGGGCCAaGGTACGCAAGTCACCGTGTCCTCT | |
| Nb2 | CAGGTGCAGCTGGTCGAGTCTGGGGGAGGATTGATGCAGGCTGGGGGCTCTCTG | 5 |
| (H11H4) | AGACTCTCCTGTGCAGTCTCTGGACGCACCTTCAGTACCGCTGCCATGGGCTGGTT | |
| | CCGCCAGGCTCCAGGGAAGGAGCGTGAGTTTGTAGCAGCTATTAGGTGGAGTGG | |
| | TGGTAGCGCATACTATGCAGACTCCGTGAAGGGCCGATTCACCATCTCCAGAGAC | |
| | AAGGCCAAGAACACGGTATATCTGCAAATGAACAGCCTGAAATATGAGGACACGGCC | |
| | GTTTATTACTGTGCACAAACGCATTATGTTTCTTATCTCCTTAGCGACTATGCCACT | |
| | TGGCCTTATGACTACTGGGGCCAGGGGACCCAGGTCACCGTCTCCTCAAAA | |

The VHH72 dimer was constructed by differentially codon-optimizing two VHH72 monomers, linked by a glycine-serine linker (GGGGS₃, SEQ ID NO:28) so that though the amino-acid sequence for the two monomers was identical, the nucleic acid sequences differed enough so that, if desired, TAG sites could be preferentially added to each monomer. These fragments, as well as fragments containing amber stop codons, were amplified with primers listed in Table 5 using touchdown PCR (M. R. Green, J. Sambrook, Touchdown Polymerase Chain Reaction (PCR). Cold Spring Harb. Protoc. 2019; doi:10.1101/pdb.top095109) and the resulting products were separated on 0.8-1.2% (w/v) agarose gels, and purified using GeneJet™ Gel Extraction Kit (ThermoFisher Scientific, USA) according to the manufacturer's instructions. Vector backbones were prepared through restriction digestion and purified through gel extraction as previously mentioned. Fragments and vector backbones were then ligated using the SLiCE cloning protocol (Y. Zhang, U. Werling, W. Edelmann, Seamless Ligation Cloning Extract (SLiCE) cloning method. Methods Mol. Biol. 1116:235-244 (2014)) transformed into chemically competent DH10B E. coli cells and selected on LB agar plates containing the appropriate antibiotic. Colonies were selected and propagated prior to purification. Genetic sequences of each plasmid were confirmed using the Sanger sequencing method. The primers and template used for PCR amplification, as well as the vector backbone and restriction enzymes used for linearization are summarized for each plasmid in Tables 3 and 5.

TABLE 5
Primers

| No. | Primer | Sequence | Size (bp) | SEQ ID NO: |
|---|---|---|---|---|
| 1 | pBAD UNI F | CCCGTTTTTTGGGCTAACAGGAG | 23 | 6 |
| 2 | pBAD bdSUMO- | CCACCAGATTCTTGCAACTGCACTTGAGAg | 45 | 7 |
| | VHH72A R1 | ccggtACCGGATCCG | | |
| 3 | pBAD bdSUMO- | CAAGTGCAGTTGCAAGAATCTGGTG | 25 | 8 |
| | VHH72A F2 | | | |
| 4 | pBAD UNI R | cccatatggtaccagctgcagatc | 24 | 9 |
| 5 | VHH72[Q1TAG] F | ATCCGGTaccggcTCTCAAGTGCAGTTGCAAG | 41 | 10 |
| | | AATCTGGTG | | |
| 6 | VHH72[Q1TAG] R | CTTGCAACTGCACTTGAGAgccggtACCGGAT | 39 | 11 |
| | | CCGCCGG | | |
| 7 | VHH72[Q13TAG]F | GGTGGCGGATTGGTCTAGGCGGGGGGTTC | 39 | 12 |
| | | GCTTCGCCTG | | |
| 8 | VHH72[Q13TAG] R | GCGAACCCCCCGCCTAGACCAATCCGCCAC | 40 | 13 |
| | | CAGATTCTTG | | |
| 9 | VHH72[G42TAG]F | CCGCCAGGCTCCGTAGAAAGAACGTGAAT | 40 | 14 |
| | | TCGTTGCGACC | | |
| 10 | VHH72[G42TAG] R | CGAATTCACGTTCTTTCTACGGAGCCTGGC | 41 | 15 |
| | | GGAACCAGCCC | | |
| 11 | pBAD-VHH72 F1 | GGGCTAACAGGAGGAATTAACCATGGGCC | 54 | 16 |
| | | AAGTGCAGTTGCAAGAATCTGGTGG | | |
| 12 | pBADVHH72A[G129 | CAGACTGGAAGTACAGGTTTTCACCctaaccT | 56 | 17 |
| | TAG]-TEV R | GAACTAACTGTTACCTGCGTGCCC | | |
| 13 | pBAD-VHH72A- | GGTGAAAACCTGTACTTCCAGTCTGGCTCC | 57 | 18 |
| | TEVGFP F2 | GTTAGCAAAGGTGAAGAACTGTTTACC | | |
| 14 | pBAD-bdSUMO- | GACTCGACCAGCTGCACCTGAGAgccggtACC | 50 | 19 |
| | H11H4 R1 | GGATCCGCCGGTCTGATG | | |
| 15 | pBAD-bdSUMO- | CATCAGACCGGCGGATCCGGTaccggcTCTCA | 50 | 20 |
| | H11H4 F2 | GGTGCAGCTGGTCGAGTC | | |
| 16 | H11H4[G42TAG] F | CCGCCAGGCTCCAtaGAAGGAGCGTGAGTT | 36 | 21 |
| | | TGTAGC | | |
| 17 | H11H4[G42TAG] R | CAAACTCACGCTCCTTctaTGGAGCCTGGCG | 39 | 22 |
| | | GAACCAGC | | |
| 18 | pBAD-H11H4-TEV F1 | CCCGTTTTTTGGGCTAACAGGAGGAATTAA | 58 | 23 |
| | | CCATGTCTCAGGTGCAGCTGGTCGAGTC | | |
| 19 | pBAD-H11H4-TEV | CAGACTGGAAGTACAGGTTTTCACCTTTTG | 51 | 24 |
| | R1 | AGGAGACGGTGACCTGGGTCC | | |
| 20 | pBAD-TEV-GFP F2 | GGTGAAAACCTGTACTTCCAGTCTGGCTCC | 57 | 25 |
| | | GTTAGCAAAGGTGAAGAACTGTTTAC | | |

### Protein Expression

Approximately 50 ng of each plasmid was combined with BL21(ai) E. coli cells. For the expression of Tet-Nbs, each TAG site containing expression plasmid was co-transformed with a GCE-machinery plasmid (Table 3, Plasmid 1 (H. S. Jang, S. Jana, R. J. Blizzard, J. C. Meeuwsen, R. A. Mehl, Access to Faster Eukaryotic Cell Labeling with Encoded Tetrazine Amino Acids. J. Am. Chem. Soc. 142:7245-7249 (2020))). The mixture was incubated for approximately 30 minutes prior to transformation on ice, and then was incubated at 42° C. for 45 seconds. Freshly transformed cells were immediately resuscitated in 2×YT media for approximately 45 minutes at 37° C. at 250 rpm, and plated on LB agar containing the proper antibiotics, and incubated overnight at 37° C. Starter cultures were inoculated by scraping a swath of cells from a fresh LB agar plate and were grown overnight (no more than 16 hours) in the presence of appropriate antibiotics. Overnight cultures were then used to inoculate expression cultures of ZY media (100 mL to 1 L) supplemented with appropriate antibiotics. For the expression of Tet3.0-Nbs, the media was also supplemented with Tet3.0 (at final concentration of 500 μM, diluted from a freshly prepared 100 mM solution solubilized in DMF). Cultures were grown with constant shaking at 275 rpm in an 126 incubator-shaker (Eppendorf, KGaA, Germany; formerly New Brunswick Scientific, USA) in baffled flasks at 37° C., until they reached an OD₆₀₀ of 1.5, wherein they were induced with a final concentration of 0.1% arabinose. Upon induction, the temperature was decreased to 18° C. and cultures expressed for an additional 30 hours prior to harvesting.

Protein Purification

After growth, cells were pelleted by centrifugation at 5400 rcf, resuspended in a lysis/wash buffer (50 mM Tris, 500 mM NaCl, 5 mM imidazole, pH 7.5) and lysed using a M-110P Microfluidizer™ system (Microfluidics Corp, USA) set at 18,000 psi. Cell debris was pelleted at 20,900 rcf for 25 minutes at 4° C. and clarified cell lysate was recovered. To bind His₆-tagged protein, clarified cell lysate was incubated with 500 μL of TALON cobalt NTA resin (Takara Bio, Japan) at 4° C. for 1 hr with rocking. Resin was collected and extensively washed with 50 resin bed volumes (bvs) of lysis buffer.

For constructs containing an N-terminal His-bdSUMO tag, the resin was resuspended in 3 bvs of storage buffer (50 mM Tris, 500 mM NaCl, 10% glycerol). bdSUMO protease (100 nM) was added to the resuspension, and the solution was incubated for 1 hr at room temperature with rocking. The resin was retained in a column and cleaved nanobody was collected in the flow-through. For all other constructs, bound protein was eluted from the TALON resin by incubation with five bvs of Elution Buffer (lysis buffer with 200 mM imidazole) and desalted into storage buffer using a PD-10 delating column according to the manufacturer's instructions. If cleavage of a TEV—GFP-HIS tag was required, TEV protease was added to the desalted solution and let incubate overnight at 4° C. Following incubation, cleaved protein was re-run over TALON resin to bind the cleaved tag, and the flow-through containing the Nb was collected. If necessary, the protein solution was concentrated by using a 3 kDa or 10 kDa MWCO Vivaspin spin-concentration filter (GE Health Sciences). Protein concentration was determined by absorbance at 280 nm and flash-frozen with liquid nitrogen and stored at −80° C. until needed.

### Mobility-Shift Assay

To quantify the reactivity of purified Tet3.0-Nbs, SDS-PAGE mobility shift assays were employed. Tet3.0-incorporated nanobodies (˜4 μM) were reacted with approximately 10 molar equivalents of sTCO-PEG5000 polymer for 10 minutes on ice. 4×SDS sample buffer (250 mM Tris, 10% (w/v) sodium dodecyl sulfate, 50% (v/v) glycerol, 20% (v/v) β-mercaptoethanol, 0.1% (w/v) bromophenol blue, pH 6.8) was added prior to incubation at 95° C. for approximately 5 minutes. Samples were then loaded onto 15 or 17% SDS-PAGE gels and run at 200 V for approximately 60 minutes, followed by Coomassie staining (50% (v/v) methanol, 40% (v/v) water, 10% (v/v) acetic acid, 0.02% (w/v) Coomassie G250) for approximately 20 minutes followed by an approximately 20 minute soak in destain solution (50% (v/v) methanol, 40% (v/v) water, 10% (v/v) acetic acid).

Tet3.0-sTCO Conjugation Reactions

To Tet3.0-containing nanobodies (150-200 μM), sTCO-functionalized PEG linkers (solubilized in DMSO to a final concentration of 10 mM or 1 mM) were slowly mixed in reaction buffer (50 mM Tris, 500 mM NaCl, 10% glycerol, pH 7.5) for a total reaction volume of 500 μL. The ratio of sTCO-PEG linker to Tet3.0-Nb was experimentally determined (FIG. 10). The reaction proceeded for 10-30 minutes at room temperature, after which the resulting products were centrifuged at 21,000 rcf for 15 minutes to remove any precipitation products before gel-filtration.

Size Exclusion Chromatography

To purify Nb complexes, homodimeric conjugation products were gel-filtered with a size exclusion column Superdex 200 16/60 (GE Healthcare) and heterodimeric and trimeric conjugation products were gel-filtered with Superdex 75 10/300 size exclusion column (GE Healthcare). The columns were equilibrated in 2 column volumes (CV) of reaction buffer, and 500 μL of clarified reaction material was injected onto the columns. The elution of the desired material was tracked by monitored by tracking the A280, and eluted fractions were analyzed with SDS-PAGE (Supplemental). The purified proteins were collected, concentrated, frozen in liquid nitrogen, and stored at −80° C. for subsequent experimentation.

Mass Spectrometry

Nanobody samples were desalted into LC-MS grade water with NAPS columns according to manufacturer's instructions. The nanobody samples were diluted to 10 μM in 15% acetonitrile and 0.1% formic acid before analysis using an Agilent 6545XT Q-ToF mass spectrometer. The instrument was modified with an ExD cell (e-MSion, Inc.) to enable electron capture dissociation (ECD). A custom nanoelectrospray source was used to ionize the samples. Briefly, 5 μL of sample was loaded into the back of a 10 cm borosilicate glass capillary (Sutter Instrument Co.) with an OD of 1 mm and an ID of 0.78 mm. The tip was loaded into the capillary holder with a platinum wire placed into the solution. The capillary holder with the emitter was positioned in front of the instrument inlet and ionized with a potential of 1200V. The instrument was operating in the 2 GHz high-resolution mode during analysis. The mass range for MS1 and MS2 spectra was 120-3200 m/z. Intact masses were determined using the Agilent MassHunter BioConfirm Deconvolution algorithm and the fragmentation data were analyzed with the ExD Viewer (Version 4.1.13). Peptide fragment ions were identified by considering the similarity of the theoretical mass to charge ratio and the isotopic cluster intensities. The resulting matches were manually curated to ensure quality of assignments.

Biolayer Interferometry (BLI)

BLI measurements were made on a fortéBIO (Menlo Park, CA) Octet™ Red96 system using anti-human capture (AHC) sensors, which are immobilized with anti-human Fc antibodies. Assays were performed in 96-well microplates at 30° C. All sample volumes were 200 μL. AHC tips were conditioned by three rounds of incubation in 10 mM glycine, pH 1.7 for 5 seconds followed by incubation in running buffer (10 mM HEPES, pH 7.6, 150 mM NaCl, 3 mM EDTA, 0.005% Tween, 1 mg/mL BSA) for 5 seconds. Conditioning was followed by baseline establishment in running buffer for 150 seconds and loading with SARS-CoV-2-RBD tagged with a C-terminal Fc tag (ACRO Biosystems, 0.005 μg/μL) for 95 seconds). After loading SARS-CoV-2-RBD-Fc onto AHC sensors, a second baseline was established in running buffer (150 seconds) and association with VHH12 nanobody variants (2 nM-1500 nM for global fits; see Table 6) was carried out in the same buffer for 200 seconds. Dissociation was subsequently measured in the same buffer for 700 seconds.

TABLE 6
Concentrations (nM) of Nbs used in BLI assays

| | | | | (Nb1- | (Nb1_{N}- | | |
|---|---|---|---|---|---|---|---|
| (Nb1_{N})₂ | (Nb1₁₃)₂ | (Nb1₄₂)₂ | (Nb1_{C})₂ | Nb1) | Nb2₄₂) | (Nb1_{N})₂ | Nb1-Nb1-Nb1 |
| 50.0 | 400.0 | 102.0 | 164.0 | 400.0 | 100.0 | 100.0 | 94.0 |
| 25.0 | 200.0 | 64.0 | 102.0 | 200.0 | 50.0 | 50.0 | 47.0 |
| 12.5 | 100.0 | 40.0 | 64.0 | 100.0 | 25.0 | 25.0 | 23.4 |
| 6.3 | 50.0 | 25.0 | 40.0 | 50.0 | 12.5 | 12.5 | 11.7 |
| 3.2 | 25.0 | 15.6 | 25.0 | 25.0 | 6.3 | 6.3 | |

Statistical Analysis of BLI Fits

Data were reference-subtracted and aligned with each other in the Octet™ Data Analysis software (ForteBio). Sensograms were fit with a 1:2 (bivalent analyte) binding model to obtain kinetic binding constants for homodimers, and a 1:1 binding model to obtain kinetic binding constants for heterodimer and trimer. Equilibrium dissociation constant (K_{D}) values were calculated from the ratio of K_{off} to Kₒₙ. Global fits with an R2 higher than 0.98 were considered acceptable.

Pseudovirus Neutralization Assay

The pseudovirus neutralization assay was performed as described previously (J. Kim, A. Mukherjee, D. Nelson, A. Jozic, G. Sahay, Rapid generation of circulating and mucosal decoy ACE2 using mRNA nanotherapeutics for the potential treatment of SARS-CoV-2. bioRxiv, doi: https://doi.org/10.1101/2020.07.24.205583 (2020)) with the following changes. 293T-hACE2 cells were seeded into white 96-well plates at 1×10⁴ cells/well and grown for 24 h. Nanobodies were serially diluted into storage buffer (30 mM Tris pH 7.5, 500 mM NaCl, 10% glycerol) to 10× the test concentration. Pseudovirus was added to each to dilution to make 1× test concentration. These were incubated at 37° C. for 1 h. Polybrene was added as previously described. Media was removed from the 96-well plates and replaced with the pseudovirus-nanobody mixture or pseudovirus-buffer alone mixture at 150 μL/well in triplicate. After 48 h, cell viability and luciferase activity were assessed with the ONE-Glo™+Tox luciferase reporter and cell viability assay kit (Promega).

Chemical Synthesis

All purchased chemicals were used without further purification. 3-Arm-PEG15K-Amine was purchased from JenKem technology. Anhydrous dichloromethane was used after overnight stirring with calcium hydride and distillation under argon atmosphere. Thin-layer chromatography (TLC) was performed on silica 60F-254 plates. The TLC spots of alkene were charred by potassium permanganate staining. Flash chromatographic purification was done on silica gel 60 (230-400 mesh size). ¹H NMR spectra were recorded at Bruker 400 MHz and 700 MHz and ¹³C NMR spectra were recorded at 175 MHz. Coupling constants (J value) were reported in hertz. The chemical shifts were shown in ppm and are referenced to the residual nondeuterated solvent peak CDCl3 (δ=7.26 in 1H NMR, δ=77.23 in 13C NMR), CD3OD (δ=3.31 in 1H NMR, δ=49.2 in ¹³C NMR), as an internal standard. Splitting patterns of protons are designated as follows: s-singlet, d-doublet, t-triplet, q-quartet, m-multiplet, bs-broad singlet.

### Chloride salt of (S)-2-amino-3-(3-(6-methyl-1,2,4,5-tetrazin-3-yl)phenyl)propanoic acid (Tet-3.0Me)(22)

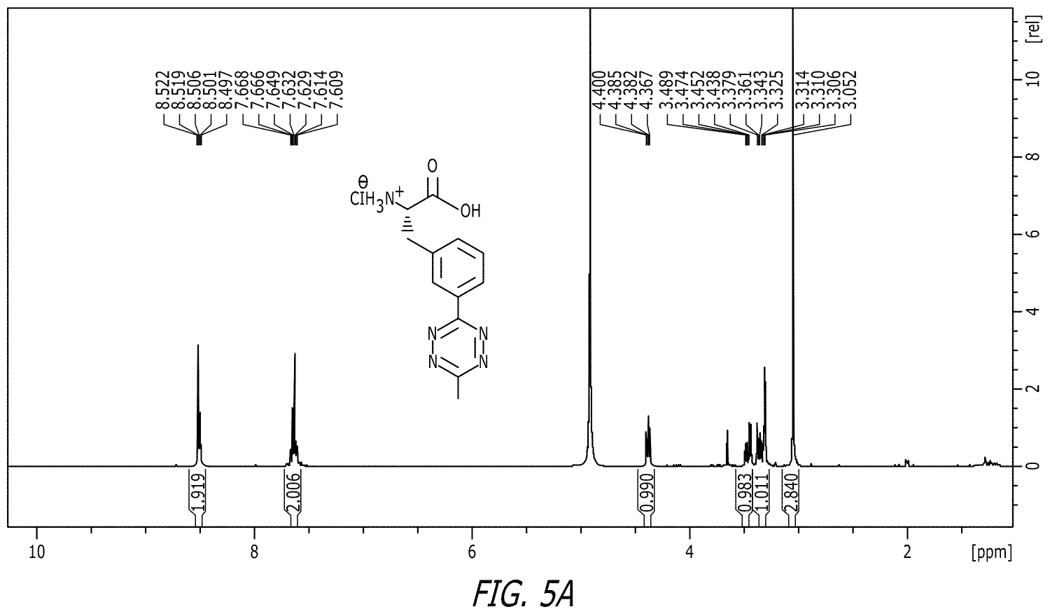

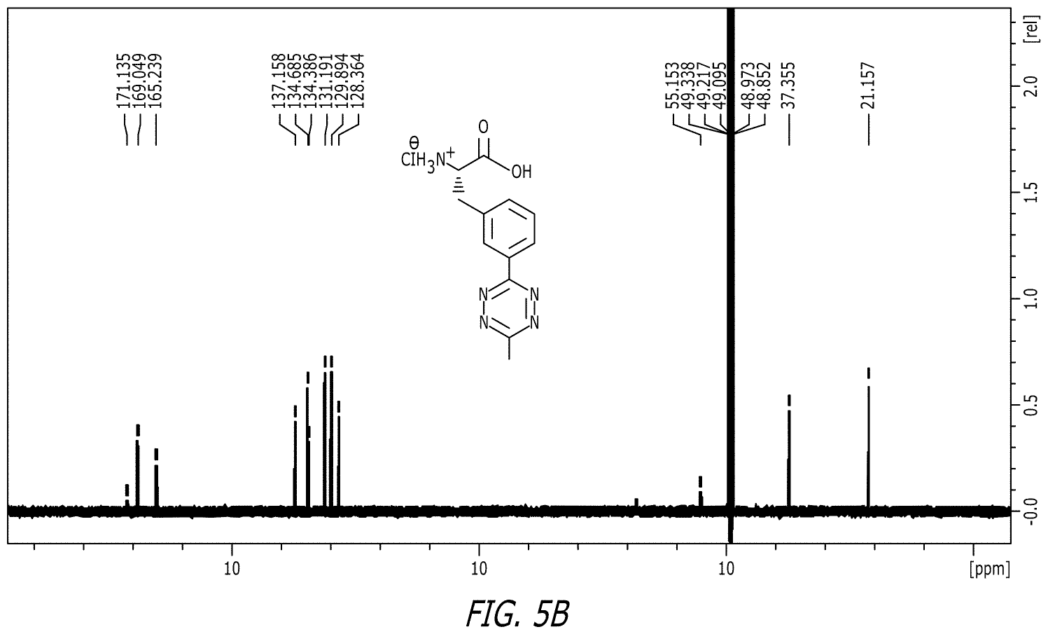

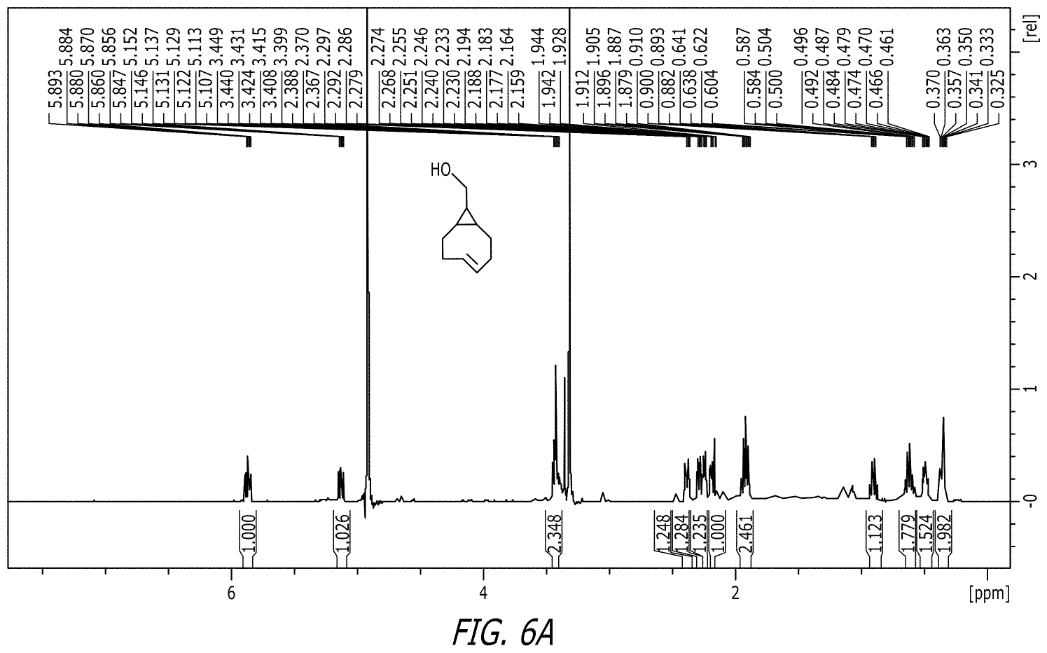

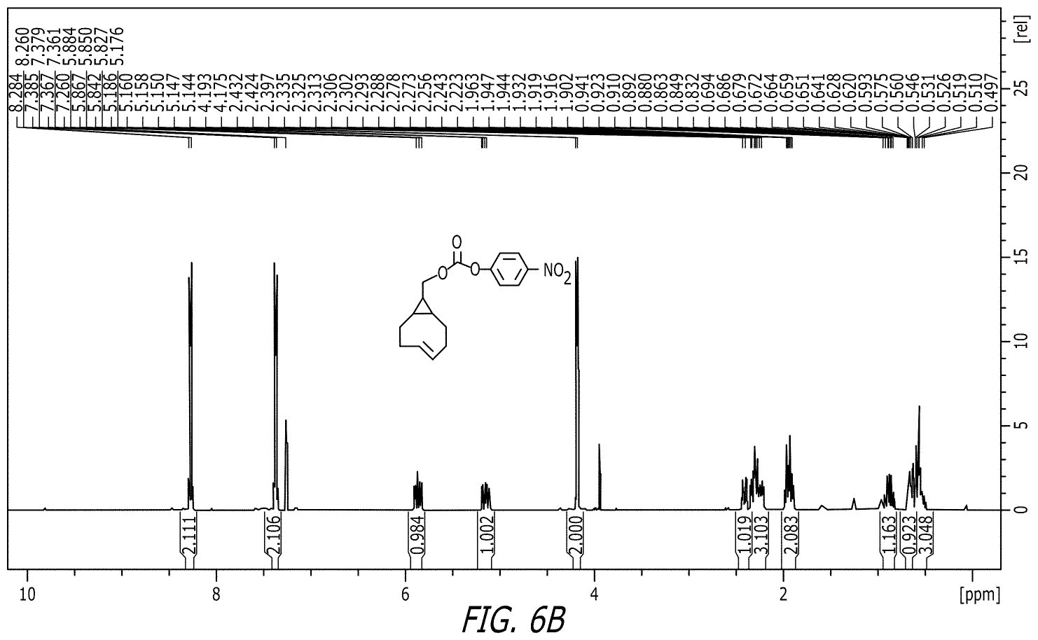

The starting material, Boc-protected 3-cyano phenylalanine (500 mg, 1.72 mmol), was added to a flame-dried, 15 mL heavy-walled reaction tube. Under argon atmosphere catalyst, Ni(OTf)₂ (306 mg, 0.86 mmol) and acetonitrile (1 mL, 18 mmol) were added to the reaction vessel. Anhydrous hydrazine (2.7 mL, 85 mmol) was then slowly added to the reaction mixture with stirring and purged with argon for 5-10 minutes, after which the tube was immediately sealed. The reaction mixture was heated to 50 degrees C. for 24 h. After that, the reaction mixture was cooled to room temperature, opened slowly and 20 eqv. 2 M NaNO₂ solution and 20 mL water were added. The aqueous phase was washed with ethyl acetate (1×20 mL) to remove the homocoupled side product. The collected aqueous phase was acidified with 4 M HCl (pH ˜2) under ice-cold conditions and extracted with ethyl acetate (3×20 mL). The combined organic layer was washed with brine, dried with anhydrous Na₂SO₄ and concentrated under reduced pressure. Silica gel flash column chromatography (30-35% ethyl acetate in hexanes with 1% acetic acid) provided 460 mg of Boc-protected-Tet-3.0Me (460 mg, 1.27 mmol) in the form of a pinkish, red gummy material. Yield 74%. ¹H NMR (400 MHz, CDCl₃) δ 8.41 (2H, t, J=7.2 Hz), 7.51-7.44 (2H, m), 5.17 (1H, d, J=7.4), 4.71 (1H, d, J=5.2), 3.33 (1H, dd, J=13.6, 5.2 Hz), 3.21 (1H, dd, J=13.2, 6.4 Hz), 3.07 (3H, s), 1.39 (9H, s). ¹³C NMR (175 MHz, CDCl₃) δ 175.6, 167.3, 164.1, 155.5, 137.7, 133.8, 132.1, 129.5, 129.1, 126.7, 80.4, 54.4, 38.1, 28.4, 21.1. The purified Boc-protected Tet-3.0 amino acid (400 mg, 1.1 mmol) was dissolved in 5 mL ethyl acetate and charged with 1 mL HCl gas-saturated 1,4 Dioxane under argon atmosphere. The reaction mixture was stirred at room temperature until the starting materials were consumed, which was confirmed by monitoring by TLC (normally 2 to 3 h). After which, the solution was concentrated under reduced pressure and re-dissolved in ethyl acetate (2×10 mL) and similarly concentrated to remove excess HCl gas resulting in a pink color solid material of Tet-3.0Me in quantitative yield (97%). ¹H NMR (400 MHz, CD₃OD, FIG. 5) δ 8.52-8.49 (2H, m), 7.66-7.60 (2H, m), 4.38 (1H, dd, J=7.2, 6 Hz), 3.46 (1H, dd, J=14.4, 5.6 Hz), 3.35 (1H, dd, J=14.4, 7.2 Hz), 3.05 (3H, s). ¹³C NMR (175 MHz, CD₃OD, FIG. 6B) δ 171.1, 169.1, 165.2, 137.1, 134.7, 134.4, 131.2, 129.8, 128.4, 55.1, 37.3, 21.1 (FIG. 12). ESI-MS calculated for C₁₂H₁₄N₅O₂ ([M+H]⁺) 260.1142, found 260.1133.

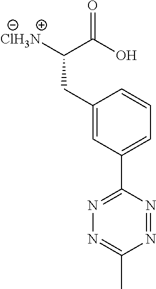

### (E)-bicyclo[6.1.0]non-4-en-9-ylmethanol (sTCO)

Synthetic procedure, ¹H NMR (700 MHz, CD3OD, Fig. S2A) δ 5.89-5.84 (1H, m), 5.15-5.10 (1H, m), 3.44-3.39 (2H, m), 2.37 (1H, d, J=13.3 Hz), 2.27 (1H, dt, J=12.6, 4.2 Hz), 2.25-2.23 (1H, m), 2.19-2.15 (1H, m), 1.94-1.87 (2H, m), 0.92-0.87 (1H, m), 0.64-0.58 (1H, m), 0.50-0.46 (1H, m), 0.37-0.31 (2H, m).

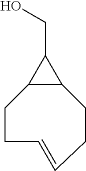 [SMILES: OCC1C2CC/C=C/CCC12]

Activated Ester-sTCO

Synthetic procedure, (H. S. Jang, S. Jana, R. J. Blizzard, J. C. Meeuwsen, R. A. Mehl, Access to Faster Eukaryotic Cell Labeling with Encoded Tetrazine Amino Acids. J. Am. Chem. Soc. 142:7245-7249 (2020).) ¹H NMR (400 MHz, CDCl₃, Fig. S2B) (δ 8.27 (2H, d, J=9.6 Hz), 7.37 (2H, d, J=9.6 Hz), 5.88-5.82 (1H, m), 5.18-5.14 (1H, m), 4.18 (2H, d, J=7.2 Hz), 2.43-2.39 (1H, m), 2.35-2.22 (3H, m), 1.96-1.90 (2H, m), 0.94-0.83 (1H, m), 0.69-0.64 (1H, m), 0.62-0.49 (3H, m).

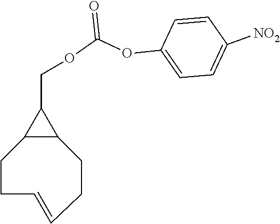 [SMILES: O=C(OCC1C2CC/C=C/CCC21)Oc1ccc([N+](=O)[O-])cc1]

PEG550 Linked di-sTCO

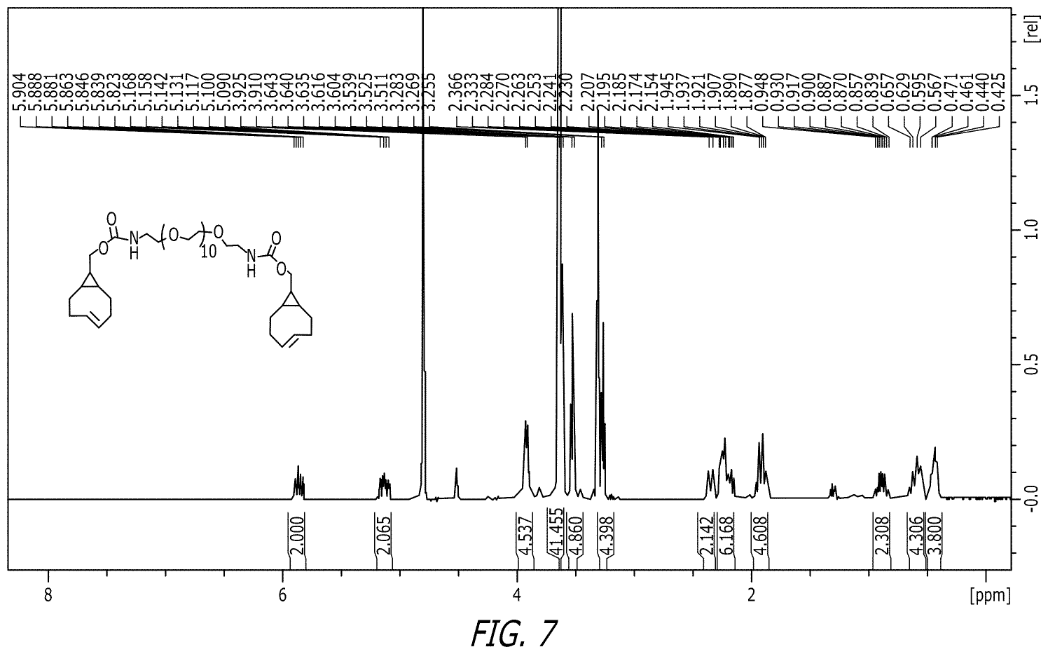

In 3 mL anhydrous dichloromethane, Amino-PEG550-Amine (40 mg, 0.072 mmol), the activated ester of sTCO (80 mg, 0.254 mmol) and followed by triethylamine (30 μL, 0.218 mmol) were added under argon atmosphere. The reaction mixture was stirred at room temperature for 24 hours. Subsequently, the solvent was concentrated onto silica gel under reduced pressure and the desired molecule was purified (40 mg, 0.042 mmol) by silica gel flash column chromatography (10-15% methanol in dichloromethane). Yield 58%. ¹H NMR (400 MHz, CD₃OD, FIG. 7) δ 5.90-5.82 (2H, m), 5.16-5.09 (2H, m), 3.91 (4H, d, J=6.0 Hz), 3.65-3.61 (40H, m), 3.52 (4H, t, J=5.6 Hz), 3.26 (4H, t, J=5.6 Hz), 2.36-2.33 (2H, m), 2.28-2.14 (6H, m), 1.94-1.87 (4H, m), 0.94-0.83 (2H, m), 0.65-0.56 (4H, m), 0.47-0.42 (4H, m).

3-Arm-PEG15k Linked Tri-sTCO

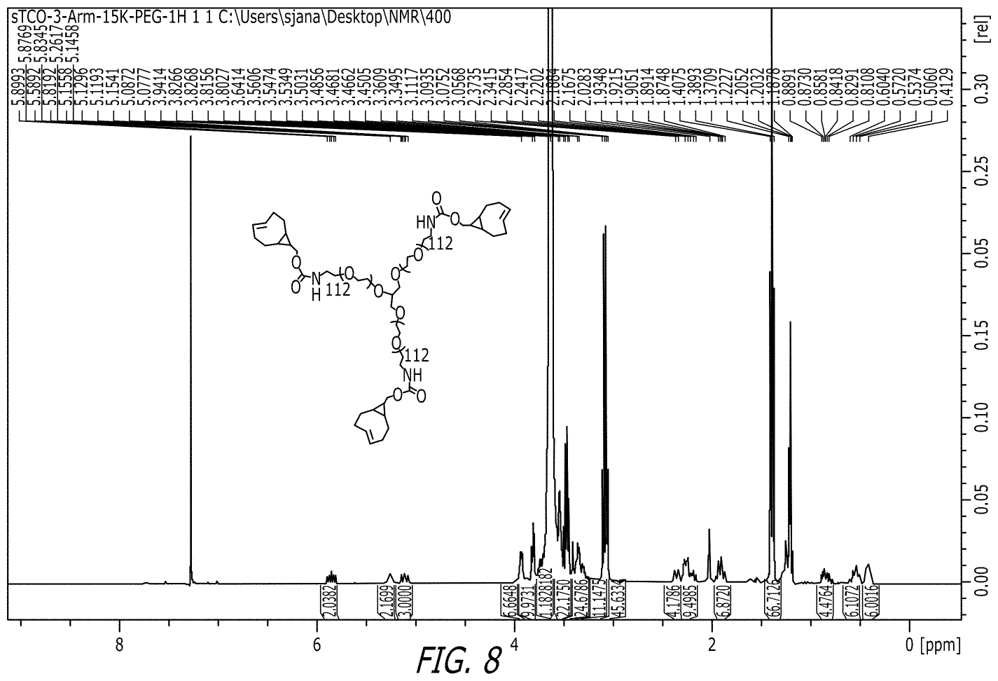

Following a similar procedure, using 50 mg of 3-Arm-PEG15K-Amine (0.004 mmol), 6 mg of the activated ester of sTCO (0.02 mmol) and 30 μL of triethylamine produced 28 mg of the title molecule (0.002 mmol) purified by silica gel flash column chromatography (25-30% methanol in dichloromethane). Yield 55%. ¹H NMR (400 MHz, CDCl₃, FIG. 8) δ 5.89-5.81 (3H, m), 5.26 (3H, bs), 5.15-5.07 (3H, m), 3.93 (6H, d, J=6.0 Hz), 3.81 (9H, t, J=5.2 Hz), 3.64 (1182H, s), 3.54 (22H, t, J=5.2 Hz), 3.46 (24H, q, J=7.2 Hz), 3.34 (11H, d, J=4.4 Hz), 3.08 (45H, q, J=7.6 Hz), 2.37-2.34 (3H, m), 2.28-2.16 (9H, m), 1.93-1.87 (6H, m), 1.38 (66H, t, J=7.8 Hz), 0.89-0.78 (4H, m), 0.60-0.50 (6H, m), 0.46-0.40 (6H, m).

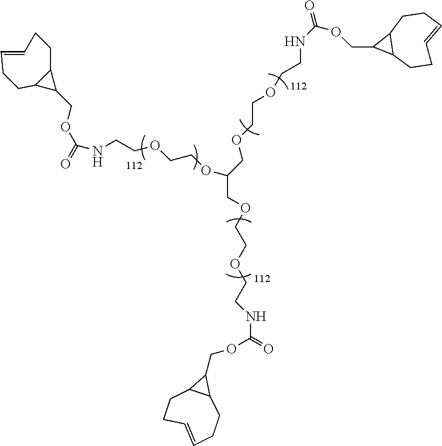

### Molecular Cloning

These fragments, as well as fragments containing amber stop codons, were amplified with primers listed in Table 5 using touchdown PCR and the resulting products were separated on 0.8-1.2% (w/v) agarose gels and purified using GeneJet™ Gel Extraction Kit (ThermoFisher Scientific, USA) according to the manufacturer's instructions. Vector backbones were prepared through restriction digestion and purified through gel extraction as previously mentioned. Fragments and vector backbones were then ligated using the SLiCE cloning protocol transformed into chemically competent DH10B E. coli cells and selected on LB agar plates containing the appropriate antibiotic. Colonies were selected and propagated prior to purification. Genetic sequences of each plasmid were confirmed using Sanger sequencing. The primers and template used for PCR amplification, as well as the vector backbone and restriction enzymes used for linearization are summarized for each plasmid in Tables 3 and 5.

Embodiment 1. A multimeric nanobody assembly, the nanobody assembly comprising: a plurality of nanobodies specific for a target ligand, wherein the nanobodies are encoded with one or more tetrazine amino acid; and a non-peptide linker comprising a strained alkene functional group associated with a tetrazine amino acid; wherein the multimeric nanobody assembly is functionalized by the covalent association of the tetrazine amino acid and the non-peptide linker such that each nanobody of the multimeric nanobody assembly retains its binding specificity to its target ligand.

Embodiment 2. The multimeric nanobody assembly of embodiment 1, wherein the non peptide linker comprises a 1,4-dihydropyridazine linkage or a dihydropyridazine linkage.

Embodiment 3. The multimeric nanobody assembly of embodiment 1 or embodiment 2, wherein the strained alkene functional group is a trans-cycloctene (TCO) functional group, or a derivative thereof.

Embodiment 4. The multimeric nanobody assembly of any one of the previous embodiments, wherein in the TCO functional group or derivative thereof, comprises a 2-headed sTCO polyethylene glycol₁₂ (PEG)₁₂ linker or a 3-armed Tri-sTCO (PEG)_{3×5k} linker.

Embodiment 5. The multimeric nanobody assembly of any one of the previous embodiments, wherein the target ligand is an antigen.

Embodiment 6. The multimeric nanobody assembly of any one of the previous embodiments, wherein the non-peptide linker is functionally associated with the nanobody assembly to form a homomultimer.

Embodiment 7. The multimeric nanobody assembly of any one of the previous embodiments, wherein the non-peptide linker is functionally associated with the nanobody assembly to form a homodimer.

Embodiment 8. The multimeric nanobody assembly of any one of the previous embodiments, wherein the non-peptide linker is functionally associated with the nanobody assembly to form a homotrimer.

Embodiment 9. The multimeric nanobody assembly of any one of the previous embodiments, wherein the non-peptide linker is functionally associated with the nanobody assembly to form a heteromultimer.

Embodiment 10. The multimeric nanobody assembly of any one of the previous embodiments, wherein the non-peptide linker is functionally associated with the nanobody assembly to form a heterodimer.

Embodiment 11. The multimeric nanobody assembly of any one of the previous embodiments, wherein the non-peptide linker is functionally associated with the nanobody assembly to form a heterotrimer.

Embodiment 11. The multimeric nanobody assembly of any one of the previous embodiments, wherein the antigen is the receptor binding domain (RBD) of the SARS-CoV-2 spike protein.

Embodiment 11. A method for inhibiting virus infectivity, the method comprising administering a therapeutically effective quantity of a pharmaceutical composition to a subject in need thereof, wherein the pharmaceutical composition comprises an active ingredient that inhibits virus infectivity and a pharmaceutically acceptable excipient, wherein the active ingredient is the multimeric nanobody assembly of any one of the previous embodiments.

Embodiment 12. The method of embodiment 11, wherein the pharmaceutical composition is formulated to be administered using a nebulizer or a nasal spray.

Embodiment 13. The method of embodiment 11 or embodiment 12, wherein the virus is a coronavirus.

Embodiment 14. The method of any one of embodiments 11-13, wherein the coronavirus is a SARS-CoV-2 coronavirus.

Embodiment 15. A method for making a multimeric nanobody assembly, the method comprising: providing at least two polynucleic acids encoding at least two nanobodies, wherein the polynucleic acids each further comprise at least one selector codon at a preselected position; providing a non-peptide linker comprising a strained alkene functional group; and providing a translational system, wherein the at least two polynucleic acids are translated by the translational system to produce the at least two nanobodies comprising a tetrazine amino acid, and wherein the translational system further includes: at least one tetrazine amino acid; an orthogonal tRNA that decodes the selector codon; an orthogonal aminoacyl-tRNA synthetase that charges the orthogonal tRNA with the tetrazine amino acid; and wherein the orthogonal tRNA inserts the tetrazine amino acid into a polypeptide in response to the selector codon, wherein the polypeptide comprises the nanobody comprising the incorporated tetrazine amino acid; and wherein the at least two least two tetrazine amino acids react with the non-peptide linker terminated by the strained alkene functional group to produce the multimeric nanobody assembly.

Embodiment 16. The method of embodiment 15, wherein the multimeric nanobody assembly is a homomultimer comprising at least two polynucleic acids each encoding nanobody VHH72 (Nb1).

Embodiment 17. The method of embodiment 15 or embodiment 16, wherein the first Nb1 polynucleic acid comprises the selector codon designed for incorporating the tetrazine amino acid at position glutamine 13 (Q13) and the second Nb1 polynucleic acid comprises the selector codon designed for incorporating the tetrazine amino acid at position glycine 42 (G42), wherein Nb1 polypeptide 1 (Nb1)₁ and Nb1 polypeptide 2 (Nb1)₂ assemble as a homodimer in the presence of the strained alkene functional group.

Embodiment 18. The method of any one of embodiments 15-17, wherein the first Nb1 polynucleic acid comprises the selector codon designed for incorporating the tetrazine amino acid at position glutamine 1 (Q1), the second Nb1 polynucleic acid comprises the selector codon designed for incorporating the tetrazine amino acid at position glutamine 1 (Q1), and the third Nb1 polynucleic acid comprises the selector codon designed for incorporating the tetrazine amino acid at position glutamine 1 (Q1), wherein Nb1 polypeptide 1 (Nb1)₁, Nb1 polypeptide 2 (Nb1)₂, and Nb1 polypeptide 3 (Nb1)₃ assemble as a homotrimer in the presence of the strained alkene functional group.

Embodiment 19. The method of any one of embodiments 15-18, wherein the multimeric nanobody assembly is a heteromultimer comprising a polynucleic acid encoding nanobody VHH72 (Nb1) and a polynucleic acid encoding nanobody H11-H4 (Nb2).

Embodiment 20. The method of any one of embodiments 15-19, wherein the Nb1 polynucleic acid comprises a selector codon designed for incorporating the tetrazine amino acid at position glutamine 1 (Q1), and the Nb2 polynucleic acid comprises a selector codon designed for incorporating the tetrazine amino acid at position glycine 42 (G42), wherein Nb1 polypeptide and Nb2 polypeptide assemble as a heterodimer in the presence of the strained alkene functional group.

Embodiment 21. The method of any one of embodiments 15-20, wherein the non-peptide linker comprises a 1,4-dihydropyridazine linkage or a dihydropyridazine linkage.

Embodiment 22. The method of any one of embodiments 15-21, wherein the strained alkene functional group is a trans-cycloctene (TCO) functional group, or a derivative thereof.

Embodiment 23. The method of any one of embodiments 15-22, wherein in the TCO or derivative thereof, comprises a 2-headed sTCO polyethylene glycol₁₂ (PEG)₁₂ linker or a 3-armed Tri-sTCO (PEG)_{3×5k} linker.

Embodiment 24. The method of any one of embodiments 15-23, wherein at least two polynucleic acids are heterologous polynucleic acids, homologous polynucleic acids, or a combination of heterologous and homologous polynucleic acids.

Embodiment 25. The method of any one of embodiments 15-24, wherein the multimeric nanobody assembly is a homodimer, wherein each monomer expresses at least one tetrazine amino acid at any position along the polypeptide sequence.

Embodiment 26. The method of any one of embodiments 15-25, wherein the multimeric nanobody assembly is a homotrimer, wherein each monomer expresses at least one tetrazine amino acid at any position along the polypeptide sequence.

Embodiment 27. The method of any one of embodiments 15-26, wherein the multimeric nanobody assembly is a heteromultimer.

Embodiment 28. The method of any one of embodiments 15-27, wherein the multimeric nanobody assembly is a heterodimer, wherein each monomer expresses at least one tetrazine amino acid at any position along the polypeptide sequence.

Embodiment 29. The method of any one of embodiments 15-28, wherein the multimeric nanobody assembly is a heterotrimer, wherein each monomer expresses at least one tetrazine amino acid at any position along the polypeptide sequence.

Embodiment 30. The method of any one of embodiments 15-29, wherein the Nb1 comprises the selector codon designed for incorporating the tetrazine amino acid at a position comprising glutamine 1(Q1), glutamine 13 (Q13), glycine 42 (G42), or serine 129 (S129).

Embodiment 31. The method of any one of embodiments 15-30, wherein the Nb2 comprises the selector codon designed for incorporating the tetrazine amino acid at a position comprising glycine 42 (G42).

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of making a multimeric nanobody assembly, the method comprising:
providing at least two polynucleotides encoding at least two nanobodies, wherein the polynucleotides each further comprise at least one selector codon at a preselected position;
providing a non-peptide linker comprising a strained alkene functional group; and
providing a translational system, wherein the at least two polynucleotides are translated by the translational system to produce the at least two nanobodies comprising a tetrazine amino acid, and wherein the translational system further includes:
at least one tetrazine amino acid;
an orthogonal tRNA that decodes the selector codon;
an orthogonal aminoacyl-tRNA synthetase that charges the orthogonal tRNA with the tetrazine amino acid; and
wherein the orthogonal tRNA inserts the tetrazine amino acid into a polypeptide in response to the selector codon,
wherein the polypeptide comprises the nanobody comprising the incorporated tetrazine amino acid; and
wherein the at least two tetrazine amino acids react with the non-peptide linker terminated by the strained alkene functional group to produce the multimeric nanobody assembly.

2. The method of claim 1, wherein the at least two polynucleotides are heterologous polynucleotides, homologous polynucleotides, or a combination of heterologous and homologous polynucleotides.

3. The method of claim 1, wherein the non-peptide linker comprises a 1,4-dihydropyridazine linkage or a dihydropyridazine linkage.

4. The method of claim 1, wherein the strained alkene functional group is a trans-cycloctene (TCO) functional group, or a derivative thereof.

5. The method of claim 1, wherein the multimeric nanobody assembly is a homomultimer.

6. The method of claim 5, wherein the multimeric nanobody assembly is a homodimer, wherein each monomer expresses at least one tetrazine amino acid at any position along the polypeptide sequence.

7. The method of claim 5, wherein the multimeric nanobody assembly is a homotrimer, wherein each monomer expresses at least one tetrazine amino acid at any position along the polypeptide sequence.

8. The method of claim 1, wherein the multimeric nanobody assembly is a heteromultimer.

9. The method of claim 8, wherein the multimeric nanobody assembly is a heterodimer, wherein each monomer expresses at least one tetrazine amino acid at any position along the polypeptide sequence.

10. The method of claim 8, wherein the multimeric nanobody assembly is a heterotrimer, wherein each monomer expresses at least one tetrazine amino acid at any position along the polypeptide sequence.

11. The method of claim 1, wherein the multimeric nanobody assembly is a homomultimer comprising at least two polynucleic acids each encoding nanobody VHH72 (Nb1).

12. The method of claim 11, wherein the Nb1 comprises the selector codon designed for incorporating the tetrazine amino acid at a position comprising glutamine 1 (Q1), glutamine 13 (Q13), glycine 42 (G42), or serine 129 (S129).

13. The method of claim 11, wherein the first Nb1 polynucleic acid comprises the selector codon designed for incorporating the tetrazine amino acid at position glutamine 13 (Q13) and the second Nb1 polynucleic acid comprises the selector codon designed for incorporating the tetrazine amino acid at position glycine 42 (G42), wherein Nb1 polypeptide 1 (Nb1)₁ and Nb1 polypeptide 2 (Nb1)₂ assemble as a homodimer in the presence of the strained alkene functional group.

14. The method of claim 1, wherein the multimeric nanobody assembly is a heteromultimer comprising a polynucleic acid encoding nanobody VHH72 (Nb1) and a polynucleic acid encoding nanobody H11-H4 (Nb2).

15. The method of claim 14, wherein the Nb1 comprises the selector codon designed for incorporating the tetrazine amino acid at a position comprising glutamine 1 (Q1), glutamine 13 (Q13), glycine 42 (G42), or serine 129 (S129) and the Nb2 comprises the selector codon designed for incorporating the tetrazine amino acid at a position comprising glycine 42 (G42).

16. The method of claim 15, wherein the Nb1 polynucleic acid comprises a selector codon designed for incorporating the tetrazine amino acid at position glutamine 1 (Q1), and the Nb2 polynucleic acid comprises a selector codon designed for incorporating the tetrazine amino acid at position glycine 42 (G42), wherein Nb1 polypeptide and Nb2 polypeptide assemble as a heterodimer in the presence of the strained alkene functional group.

17. The method of claim 4, wherein in the TCO or derivative thereof, comprises a 2-headed sTCO polyethylene glycol₁₂ (PEG)₁₂ linker or a 3-armed Tri-sTCO (PEG)_{3×5k} linker.

18. A multimeric nanobody assembly, the nanobody assembly comprising:
a plurality of nanobodies specific for a target ligand, wherein the nanobodies are encoded with one or more tetrazine amino acid; and
a non-peptide linker comprising a strained alkene functional group associated with a tetrazine amino acid;
wherein the multimeric nanobody assembly is functionalized by the covalent association of the tetrazine amino acid and the non-peptide linker such that each nanobody of the multimeric nanobody assembly retains its binding specificity to its target ligand.

19. The multimeric nanobody assembly of claim 18, wherein the multimeric nanobody assembly is a homomultimer comprising at least two polynucleic acids each encoding nanobody VHH72 (Nb1).

20. The multimeric nanobody assembly of claim 18, wherein the multimeric nanobody assembly is a heteromultimer comprising a polynucleic acid encoding nanobody VHH72 (Nb1) and a polynucleic acid encoding nanobody H11-H4 (Nb2).


SEQUENCE LISTING (28 sequences)

SEQ ID NO: 1 - DNA, 287 bp, synthetic construct
  source 1..287: mol_type = other DNA
agcgccgccggtggtgaagaagataaaaaaccggcgggcggcgaaggcggtggcgcgcatatcaacctgaaagtgaaaggtcaggatggtaacgaagtgttcttccgcattaaacgcagcacccagctgaagaaactgatgaacgcctattgcgatcgccagagcgtcgatatgaccgccattgccttcctgtttgatggccgccgtctgcgtgcggaacagacgccggacgaactggaaatggaagacggcgatgaaattgatgcgatgctgcatcagaccggcgg

SEQ ID NO: 2 - DNA, 714 bp, synthetic construct
  source 1..714: mol_type = other DNA
gttagcaaaggtgaagaactgtttaccggcgttgtgccgattctggtggaactggatggtgatgtgaatggccataaatttagcgttcgtggcgaaggcgaaggtgatgcgaccaacggtaaactgaccctgaaatttatttgcaccaccggtaaactgccggttccgtggccgaccctggtgaccaccctgacctatggcgttcagtgctttagccgctatccggatcatatgaaacgccatgatttctttaaaagcgcgatgccggaaggctatgtgcaggaacgtaccattagcttcaaagatgatggcacctataaaacccgtgcggaagttaaatttgaaggcgataccctggtgaaccgcattgaactgaaaggtattgattttaaagaagatggcaacattctgggtcataaactggaatataatttcaacagccataatgtgtatattaccgccgataaacagaaaaatggcatcaaagcgaactttaaaatccgtcacaacgtggaagatggtagcgtgcagctggcggatcattatcagcagaataccccgattggtgatggcccggtgctgctgccggataatcattatctgagcacccagagcgttctgagcaaagatccgaatgaaaaacgtgatcatatggtgctgctggaatttgttaccgccgcgggcattacccacggtatggatgaactgtataaa

SEQ ID NO: 3 - DNA, 381 bp, synthetic construct
  source 1..381: mol_type = other DNA
caagtgcagttgcaagaatctggtggcggattggtccaagcggggggttcgcttcgcctgagttgtgccgcgtctggacgcactttttctgagtacgcaatgggctggttccgccaggctccgggcaaagaacgtgaattcgttgcgaccatttcctggagcggaggctctacgtactacaccgattcggttaagggccgtttcaccatttctcgtgacaatgcgaagaatacggtttatcttcaaatgaactcattgaagccagacgatacagcggtctattactgtgctgcagcgggattaggtacggtagtgtcggaatgggattatgattattatttagattattggggccagggcacgcaggtaacagttagttca

SEQ ID NO: 4 - DNA, 384 bp, synthetic construct
  source 1..384: mol_type = other DNA
tctcaagttcaattacaggagtccgggggaggccttgtgcaagctggtggtagtttacgcctgtcttgcgcagcgtctgggcgcaccttctccgaatacgcaatgggttggttccgtcaagcacctggaaaggagcgcgagtttgttgctactatttcctggtccggcggtagtacttactatacggacagcgtcaagggccgctttacgatttcgcgtgacaacgctaaaaacaccgtgtatttacagatgaactcattaaagcccgacgatacggccgtgtattattgtgccgccgcaggcttaggtacagttgtttcagaatgggattacgactattacttagattattggggccaaggtacgcaagtcaccgtgtcctct

SEQ ID NO: 5 - DNA, 384 bp, synthetic construct
  source 1..384: mol_type = other DNA
caggtgcagctggtcgagtctgggggaggattgatgcaggctgggggctctctgagactctcctgtgcagtctctggacgcaccttcagtaccgctgccatgggctggttccgccaggctccagggaaggagcgtgagtttgtagcagctattaggtggagtggtggtagcgcatactatgcagactccgtgaagggccgattcaccatctccagagacaaggccaagaacacggtatatctgcaaatgaacagcctgaaatatgaggacacggccgtttattactgtgcacaaacgcattatgtttcttatctccttagcgactatgccacttggccttatgactactggggccaggggacccaggtcaccgtctcctcaaaa

SEQ ID NO: 6 - DNA, 23 bp, synthetic construct
  source 1..23: mol_type = other DNA
cccgttttttgggctaacaggag

SEQ ID NO: 7 - DNA, 45 bp, synthetic construct
  source 1..45: mol_type = other DNA
ccaccagattcttgcaactgcacttgagagccggtaccggatccg

SEQ ID NO: 8 - DNA, 25 bp, synthetic construct
  source 1..25: mol_type = other DNA
caagtgcagttgcaagaatctggtg

SEQ ID NO: 9 - DNA, 24 bp, synthetic construct
  source 1..24: mol_type = other DNA
cccatatggtaccagctgcagatc

SEQ ID NO: 10 - DNA, 41 bp, synthetic construct
  source 1..41: mol_type = other DNA
atccggtaccggctctcaagtgcagttgcaagaatctggtg

SEQ ID NO: 11 - DNA, 39 bp, synthetic construct
  source 1..39: mol_type = other DNA
cttgcaactgcacttgagagccggtaccggatccgccgg

SEQ ID NO: 12 - DNA, 39 bp, synthetic construct
  source 1..39: mol_type = other DNA
ggtggcggattggtctaggcggggggttcgcttcgcctg

SEQ ID NO: 13 - DNA, 40 bp, synthetic construct
  source 1..40: mol_type = other DNA
gcgaaccccccgcctagaccaatccgccaccagattcttg

SEQ ID NO: 14 - DNA, 40 bp, synthetic construct
  source 1..40: mol_type = other DNA
ccgccaggctccgtagaaagaacgtgaattcgttgcgacc

SEQ ID NO: 15 - DNA, 41 bp, synthetic construct
  source 1..41: mol_type = other DNA
cgaattcacgttctttctacggagcctggcggaaccagccc

SEQ ID NO: 16 - DNA, 54 bp, synthetic construct
  source 1..54: mol_type = other DNA
gggctaacaggaggaattaaccatgggccaagtgcagttgcaagaatctggtgg

SEQ ID NO: 17 - DNA, 56 bp, synthetic construct
  source 1..56: mol_type = other DNA
cagactggaagtacaggttttcaccctaacctgaactaactgttacctgcgtgccc

SEQ ID NO: 18 - DNA, 57 bp, synthetic construct
  source 1..57: mol_type = other DNA
ggtgaaaacctgtacttccagtctggctccgttagcaaaggtgaagaactgtttacc

SEQ ID NO: 19 - DNA, 50 bp, synthetic construct
  source 1..50: mol_type = other DNA
gactcgaccagctgcacctgagagccggtaccggatccgccggtctgatg

SEQ ID NO: 20 - DNA, 50 bp, synthetic construct
  source 1..50: mol_type = other DNA
catcagaccggcggatccggtaccggctctcaggtgcagctggtcgagtc

SEQ ID NO: 21 - DNA, 36 bp, synthetic construct
  source 1..36: mol_type = other DNA
ccgccaggctccatagaaggagcgtgagtttgtagc

SEQ ID NO: 22 - DNA, 39 bp, synthetic construct
  source 1..39: mol_type = other DNA
caaactcacgctccttctatggagcctggcggaaccagc

SEQ ID NO: 23 - DNA, 58 bp, synthetic construct
  source 1..58: mol_type = other DNA
cccgttttttgggctaacaggaggaattaaccatgtctcaggtgcagctggtcgagtc

SEQ ID NO: 24 - DNA, 51 bp, synthetic construct
  source 1..51: mol_type = other DNA
cagactggaagtacaggttttcaccttttgaggagacggtgacctgggtcc

SEQ ID NO: 25 - DNA, 56 bp, synthetic construct
  source 1..56: mol_type = other DNA
ggtgaaaacctgtacttccagtctggctccgttagcaaaggtgaagaactgtttac

SEQ ID NO: 26 - protein, 5 aa, synthetic construct
SGTGS

SEQ ID NO: 27 - protein, 126 aa, synthetic construct
VQLQESGGGLVQAGGSLRLSCAASGRTFSEYAMGWFRQAPGKEREFVATISWSGGSTYYTDSVKGRFTISRDNAKNTVYLQMNSLKPDDTAVYYCAAAGLGTVVSEWDYDYYLDYWGQGTQVTVSS

SEQ ID NO: 28 - protein, 5 aa, synthetic construct
GGGGS